(19) United States
(12) Patent Application Publication
(10) Pub. No.: US 2024/0289586 A1
(43) Pub. Date: Aug. 29, 2024

(54) DIAGNOSTIC DATA FEEDBACK LOOP AND METHODS OF USE THEREOF

(71) Applicant: Freenome Holdings, Inc., South San Francisco, CA (US)

(72) Inventors: Sanjeev BALAKRISHNAN, San Francisco, CA (US); Marvin BERTIN, San Francisco, CA (US); Richard BOURGON, San Francisco, CA (US); William DANFORTH, San Francisco, CA (US); Matthew MAHOWALD, San Francisco, CA (US); Charles Edward Selkirk ROBERTS, Topanga, CA (US)

(21) Appl. No.: 18/533,328

(22) Filed: Dec. 8, 2023

Related U.S. Application Data
(63) Continuation of application No. PCT/US2022/032654, filed on Jun. 8, 2022.
(60) Provisional application No. 63/209,252, filed on Jun. 10, 2021.

(51) Int. Cl. G06N 3/04; G06N 3/08

(52) U.S. Cl. CPC G06N 3/04; G06N 3/08

(57) ABSTRACT

The present disclosure provides methods and systems for using diagnostic feedback loops as an integrated mechanism to generate or improve machine learning classification models by using biological sample molecular data, clinical data, controlled experimental data, real-world clinical data, or a combination thereof. The feedback loops described herein may receive a combination of biological sample molecular data, clinical data, controlled experimental data, and real-world clinical data which may be processed using the feedback loop, and a machine learning classifier may be generated or revised as an output of the feedback loop. Applications of the diagnostic feedback loops may include disease screening, diagnosis, prognosis, and treatment determination.

### CROSS-REFERENCE TO RELATED APPLICATIONS

This application is a continuation of International Patent Application No. PCT/US2022/032654, filed Jun. 8, 2022, which claims the benefit of U.S. Provisional Patent Application No. 63/209,252, filed Jun. 10, 2021, each of which is incorporated by reference herein in its entirety.

### FIELD

The present disclosure relates generally to a data feedback loop system and method of use thereof to refine classification models for disease screening, diagnosis, detection, prognosis, and therapy response.

### BACKGROUND

A primary issue for any screening tool may be the compromise between false positive and false negative results (or specificity and sensitivity) which lead to unnecessary investigations in the former case, and ineffectiveness in the latter case. One important characteristic of a valuable screening test is a high Positive Predictive Value (PPV), minimizing unnecessary investigations but detecting the vast majority of disease. Blood-based screening approaches for disease based on circulating analytes provides an opportunity to minimize unnecessary investigations, but the sample-developed test accuracy when applied to a general population may not reflect the population incidence of a disease with complete accuracy.

Machine learning models may be used to classify individuals in a population for disease screening, diagnosis, prognosis, or treatment decisions. While statistical methods guide and inform adequate generation of classification models, an accuracy gap may exist when applied to the general population. The accuracy gap between test sample data used to train a model and data derived when deployed to a general population may provide challenges to health care professionals trying to make effective monitoring and treatment decisions with imperfect information.

### SUMMARY

Methods and systems are provided to augment diagnostic discovery platforms with real-world data (RWD) and refine existing classification models to catalyze advances in the field of medical screening and diagnosis. The present disclosure provides methods and systems directed to an information feedback loop useful for generating or improving classification models. In some embodiments, the methods and systems may be useful for generating or improving classification models of disease detection, diagnosis, prognosis, and to inform treatment decisions for individuals. Elements and features of the information feedback loops may be automated to continuously refine existing models, or used to support generation of new classification models based on input from RWD.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

In one aspect, provided herein is a data feedback loop comprising:
a research platform module that trains or re-trains a classification model;
a production module produces input data, wherein the production module comprises the classification model deployed for use in a population; and
an external feedback/data collection module that receives data from real-world execution of the classification model, and is operatively linked to the research platform module.

In one embodiment, the data feedback loop further comprises an evaluation environment module that monitors and evaluates validated models for deployment, wherein the evaluation environment module is operatively linked between the research platform module and the production module.

In one embodiment, the research platform module and the evaluation environment module analyze molecular or clinical data from an individual, wherein the data is de-identified of any identifying features of the individual.

In one embodiment, the evaluation environment module further comprises an evaluation/deployment module that provides productionizing of a validated model to prepare for deployment.

In one embodiment, the data feedback loop further comprises a data ingestion module that ingests data, wherein the data ingestion module is operatively linked between the external feedback/data collection module and the research platform module.

In one embodiment, the research platform module provides automatic featurization of input data that meet predetermined specifications and automatic processing of featurized input data using a machine learning pipeline element within the research platform module.

In one embodiment, the machine learning pipeline element comprises a model selection and locking element, and a model research validation element.

In one embodiment, the production module and the external feedback/data collection module analyze molecular or clinical data from an individual, wherein the data is de-identified of any identifying features of the individual, wherein the data is de-identified of any identifying features of the individual before the data is ingested into the research platform module.

In one embodiment, the production module and/or external feedback/data collection module receives molecular or clinical data from an individual and processes the data via the research platform module, wherein the data is de-identified of any identifying features of the individual before the data is ingested into the research platform module.

In one embodiment, the external feedback/data collection module receives clinical metadata or labels associated with additional disorders, symptoms, or diseases, and matches the clinical metadata or labels to the molecular data obtained from the individual before processing via the research platform module.

In one embodiment, the research platform module further comprises a cohort selection training/retraining module that selects classes of training samples for the classification module or re-trains the classification model.

In one embodiment, the evaluation environment module comprises an evaluation/deployment module that provides productionizing of a validated model received from the research platform module to prepare for the validated model for deployment, and the evaluation/deployment module is operatively linked between the cohort selection and retraining module and the product inference module.

In one embodiment, the production module further comprises a product inference module that produces raw data for ingestion into the data feedback loop system, and a research ingestion module that processes clinical metadata or labels with quality control metrics, and matches the clinical metadata with patient molecular data.

In one embodiment, the external feedback/data collection module pushes the matched clinical and molecular data to the research platform module.

In one aspect, provided herein is a data feedback loop comprising:
a cohort selection and retraining module that selects classes of training samples for a classification model or re-trains the classification model;
a product inference module that produces raw data for ingestion into the data feedback loop system; and
an external feedback/data collection module that receives data from real-world execution of the classification model.

In one embodiment, the external feedback/data collection module is operatively linked to the cohort selection and retraining module.

In one embodiment, the cohort selection and retraining module further comprises a training module that trains the classification model.

In one embodiment, the classification model is trained using a federated learning approach.

In one embodiment, the classification model is trained using an active learning approach.

In one embodiment, the data feedback loop comprises an evaluation/deployment module that productionizes a validated model to prepare for deployment, and the evaluation/deployment module is operatively linked between the cohort selection and retraining module and the product inference module.

In one embodiment, the data feedback loop comprises directional information flow from the evaluation/deployment module to the product inference module and either: a) back to the evaluation/deployment module or b) forward to the external feedback/data collection module or the cohort selection and retraining module.

In one embodiment, data flows from the evaluation/deployment module to the product inference module and back to the evaluation/deployment module or forward to the external feedback/data collection module.

In one embodiment, the evaluation/deployment module further comprises: 1) an input selected from: a) a validated model, b) gold standard data sets, c) de-identified molecular data, d) de-identified clinical data, and e) a combination thereof; and 2) an output of a deployed validated classification model.

In one embodiment, the data feedback loop comprises directional information flow from the evaluation/deployment module to the product inference module and forward to the cohort selection and retraining module without an external feedback/data collection module.

In one embodiment, data flows from the cohort selection and retraining module to the product inference module to the external feedback/data collection module and back to the cohort selection and retraining module.

In one embodiment, the cohort selection and retraining module further comprises: 1) an input selected from a) de-identified patient data matched with a sample, b) feedback loop batching specifications, c) ingested data quality specifications, and d) a combination thereof; and 2) an output of a validated classification model.

In one embodiment, the data feedback loop further comprises a data ingestion module that ingests data, and the data ingestion module is operatively linked between the external feedback/data collection module and the cohort selection and retraining module.

In one embodiment, the data feedback loop further comprises a research ingestion module that processes clinical metadata or labels with quality control metrics, matches the clinical metadata with patient molecular data, or pushes the matched clinical metadata and molecular data to the research platform module, wherein the research ingestion module is operatively linked between the external feedback/data collection module and the cohort selection and retraining module.

In one embodiment, the research ingestion module further comprises: 1) an input selected from: a) processed sample molecular data, b) disease and clinical condition labels, c) clinical data, and d) a combination thereof; and 2) an output of de-identified patient data matched with a sample.

In one embodiment, the input comprises de-identified patient data matched with a sample.

In one embodiment, the input comprises feedback loop batching specifications.

In one embodiment, the input comprises ingested data quality specifications.

In one embodiment, the data feedback loop comprises directional information flow from the cohort selection and retraining module to the product inference module to the external feedback/data collection module and back to the cohort selection and retraining module.

In one embodiment, the product inference module further comprises: 1) an input selected from a) a deployed model, b) a validated model, c) blood sample data, and d) a combination thereof; and 2) an output selected from a) processed sample molecular data, b) patient test results, c) patient metadata, d) de-identified labeled patient sample data, e) de-identified sample molecular data, and f) a combination thereof.

In one embodiment, the cohort selection training/retraining model comprises: 1) inputs selected from a) de-identified patient data matched with a sample, b) feedback loop batching specifications, c) ingested data quality specifications, and d) a combination thereof; and 2) an output of a validated classification model.

In one embodiment, the input is de-identified patient data matched with a biological sample from the same patient.

In one embodiment, the de-identified patient data is clinical data, electronic medical record (EMR) data, patient metadata, or patient molecular data.

In one embodiment, the input is feedback loop batching specifications.

In one embodiment, the input is ingested data quality specifications.

In one embodiment, the evaluation/deployment module comprises: 1) inputs selected from: a) a validated model, b) gold standard data sets, c) de-identified molecular data, d) de-identified clinical data, and e) a combination thereof; and 2) an output of a deployed validated classification model.

In one embodiment, the research ingestion module comprises: 1) inputs selected from a) processed sample molecular data, b) disease and clinical condition labels, c) clinical data, and d) a combination thereof; and 2) an output of de-identified patient data matched with a sample to the research platform module.

In one aspect, provided herein is a classification model comprising a data feedback loop, wherein the data feedback loop comprises:
a cohort selection and retraining module that selects classes of training samples for a classification model or re-trains the classification models;
a product inference module that produces raw data for ingestion into the data feedback loop system; and
an external feedback/data collection module that receives data from real-world execution of the classification model, wherein the external feedback/data collection module is operatively linked to a research platform module.

In one embodiment, the data feedback loop system further comprises an evaluation/deployment module that productionizes a validated model to prepare for deployment, wherein the evaluation/deployment module is operatively linked between the cohort selection and retraining module and the product inference module.

In one embodiment, the data feedback loop system further comprises a data ingestion module that ingests data, wherein the data ingestion module is operatively linked between the external feedback/data collection module and the cohort selection and retraining module.

In one embodiment, the classification model comprises a machine learning classifier.

In some examples, the classification model is trained using a federated learning approach.

In some examples, the classification model is trained using an active learning approach.

In some examples, the machine learning classifier comprises a cancer risk stratification model classifier.

In some examples, the machine learning classifier comprises a lung, breast, prostate, pancreatic, liver, or colorectal cancer risk stratification model classifier.

In some examples, the machine learning classifier comprises a colorectal cancer risk stratification model classifier.

In one embodiment, the data feedback loop comprises an evaluation/deployment module that productionizes a validated model to prepare for deployment, wherein the evaluation/deployment module is operatively linked between the cohort selection and retraining module of the research platform module and the product inference module of the production module.

In one embodiment, the data feedback loop comprises a data ingestion module that ingests data, wherein the data ingestion module is operatively linked between the external feedback/data collection module and the cohort selection and retraining module.

In another aspect, provided herein is a method for improving a diagnostic classifier model, the method comprising:
a) obtaining molecular and/or clinical data from an individual sample associated with the presence or absence of a specified property of a disease or disorder requiring classification;
b) processing the molecular and/or clinical data from the individual using a data feedback loop comprising:
   i) a research platform module that trains or re-trains a diagnostic classifier;
   ii) a production module that produces input data, wherein the production module comprises the diagnostic classifier; and
   iii) an external feedback/data collection module that receives data from real-world execution of the diagnostic classifier; and
c) re-training the diagnostic classifier to improve one or more classification metrics of the diagnostic classifier.

In one embodiment, the classifier model comprises a machine learning classifier.

In some examples, the machine learning classifier is trained using a federated learning approach.

In some examples, the machine learning classifier is trained using an active learning approach.

In some examples, the machine learning classifier comprises a cancer risk stratification model classifier.

In some examples, the machine learning classifier comprises a lung, breast, prostate, pancreatic, liver, or colorectal cancer risk stratification model classifier.

In some examples, the machine learning classifier comprises a colorectal cancer risk stratification model classifier.

In one embodiment, the machine learning classifier is trained on a set of training biological samples, wherein the set of training biological samples consists of a first subset of the training biological samples identified as having the specified property and a second subset of the training biological samples identified as not having the specified property, and the machine learning classifier provides an output classification of whether the biological sample has the specified property, thereby distinguishing a population of individuals having the specified property.

In various examples, the specified property can be a clinically-diagnosed disorder.

In various examples, the clinically-diagnosed disorder is cancer. As examples, the cancer can be colorectal cancer, liver cancer, lung cancer, pancreatic cancer, or breast cancer.

In some examples, the specified property is a clinical stage of the disease or disorder.

In some examples, the specified property is responsiveness to a treatment for the disease or disorder.

In one example, the specified property comprises a continuous measurement of a patient trait or phenotype at two or more points in time.

In one embodiment, the data feedback loop further comprises an evaluation/deployment module that productionizes a validated model to prepare for deployment, wherein the evaluation/deployment module is operatively linked between the research platform module and the production module.

In various embodiments, the research platform module comprises: i) processing input data, and ii) changing weights of the features of a classification model architecture of an already-deployed champion classification model, wherein the champion classification model has the same feature architecture of a challenger classification model that is trained by the research platform module.

In various embodiments, the research platform module comprises: i) processing input data, and ii) changing both features and weights of the features of a classification model architecture of an already-deployed champion classification model.

In various embodiments, the research platform module comprises: i) processing input data, and ii) changing weights of the features of a classification model architecture of an already-deployed champion classification model, wherein the champion classification model has the same feature architecture of a challenger classification model being trained by the research platform module.

In various embodiments, the research platform module comprises: i) processing input data, and ii) changing both features and weights of the features of a classification model architecture of an already-deployed champion classification model.

In one example, data in the external feedback/data collection module is pushed with a predetermined periodicity into a data ingestion module before processing via the research platform module.

In certain embodiments, the data is de-identified in the data ingestion module before pushing into the research platform module.

In one embodiment, predetermined periodicity is selected from about 1 month, about 3 months, about 6 months, about 9 months, about 12 months, about 18 months, or about 24 months.

In one embodiment, predetermined periodicity occurs by the number of patient data profiles received by the data ingestion module. In some embodiments, the number of patient data profiles is selected from about 100 patient data profiles received, 200 patient data profiles received, 300 patient data profiles received, 400 patient data profiles received, 500 patient data profiles received, 600 patient data profiles received, 700 patient data profiles received, 800 patient data profiles received, 900 patient data profiles received, 1000 patient data profiles received, 1500 patient data profiles received, 2000 patient data profiles received, 2500 patient data profiles received, 3000 patient data profiles received, 3500 patient data profiles received, and 4000 patient data profiles received. A patient data profile may comprise a collection of clinical or molecular data specific to one patient at one point in time.

In another aspect, provided herein is a method of creating a new diagnostic classifier model, the method comprising:
a) obtaining molecular and/or clinical data from an individual associated with the presence or absence of a characteristic of a disease or disorder requiring classification;
b) processing the molecular and/or clinical data from the individual using a data feedback loop comprising:
   i) a research platform module that trains or re-trains a diagnostic classifier;
   ii) a production module that produces input data, wherein the production module comprises the diagnostic classifier; and
   iii) an external feedback/data collection module that receives data from real-world execution of the diagnostic classifier; and
c) training diagnostic classifier to provide one or more classification metrics capable of classifying a population of individuals for the disease or disorder.

In one embodiment, the classifier model comprises a machine learning classifier.

In some examples, the machine learning classifier is trained using a federated learning approach.

In some examples, the machine learning classifier is trained using an active learning approach.

In some examples, the machine learning classifier comprises a cancer risk stratification model classifier.

In some examples, the machine learning classifier comprises a lung, breast, prostate, pancreatic, liver, or colorectal cancer risk stratification model classifier.

In some examples, the machine learning classifier comprises a colorectal cancer risk stratification model classifier.

In one embodiment, the data feedback loop further comprises an evaluation/deployment module that productionizes a validated model to prepare for deployment, wherein the evaluation/deployment module is operatively linked between the research platform module and the production module.

In one embodiment, the data feedback loop system further comprises a data ingestion module that ingests data, wherein the data ingestion module is operatively linked between the external feedback/data collection module and the research platform module.

In one example, data in the external feedback/data collection module is pushed with a predetermined periodicity into a data ingestion module before processing via the research platform module.

In certain embodiments, the data is de-identified in the data ingestion module before pushing into the research platform module.

In one embodiment, predetermined periodicity is selected from about 1 month, about 3 months, about 6 months, about 9 months, about 12 months, about 18 months, or about 24 months.

In one embodiment, predetermined periodicity occurs by the number of patient data profiles received by the data ingestion module. In some embodiments, the number of patient data profiles is selected from about 100 patient data profiles received, 200 patient data profiles received, 300 patient data profiles received, 400 patient data profiles received, 500 patient data profiles received, 600 patient data profiles received, 700 patient data profiles received, 800 patient data profiles received, 900 patient data profiles received, 1000 patient data profiles received, 1500 patient data profiles received, 2000 patient data profiles received, 2500 patient data profiles received, 3000 patient data profiles received, 3500 patient data profiles received, or 4000 patient data profiles received. A patient data profile may comprise a collection of clinical or molecular data specific to one patient at one point in time.

In another aspect, provided herein is a system comprising a data feedback loop, wherein the data feedback loop comprises:
a) a research platform module that trains or re-trains a classification model;
b) a production module that produces input data, the production module comprising a classification model; and
c) an external feedback/data collection module that receives data from real-world execution of the classification model, wherein the external feedback/data collection module is operatively linked to the research platform module; and a computing device comprising a computer processor, an operating system configured to perform executable instructions, a memory, and a computer program comprising instructions executable by the computer processor to provide a computer application for executing the data feedback loop.

In one embodiment, the data feedback loop comprises an evaluation/deployment module that productionizes a validated model to prepare for deployment, wherein the evaluation/deployment module is operatively linked between the research platform module and the production module.

In one embodiment, the data feedback loop comprises a data ingestion module that ingests data, wherein the data ingestion module is operatively linked between the external feedback/data collection module and the research platform module.

In another aspect, provided herein is non-transitory computer-readable medium comprising machine-executable code that, upon execution by a computer processor, implements a method for re-training a diagnostic classifier, the method comprising:
a) obtaining molecular or clinical data from an individual sample associated with a presence or absence of a characteristic of a disease or disorder requiring classification;
b) processing the molecular or clinical data using a data feedback loop system, wherein the data feedback loop system comprises:
   i) a research platform module that trains or re-trains a diagnostic classifier;
   ii) a production module that produces input data, wherein the production module comprises the diagnostic classifier; and
   iii) an external feedback/data collection module that receives data from real-world execution of the diagnostic classifier; and
c) re-training the diagnostic classifier to improve one or more classification metrics of the diagnostic classifier.

In one embodiment, the diagnostic classifier comprises a machine learning classifier.

In one embodiment, the diagnostic classifier is trained using a federated learning approach.

In one embodiment, the diagnostic classifier is trained using an active learning approach.

In one embodiment, the data feedback loop further comprises an evaluation/deployment module productionizes a validated model to prepare for deployment, wherein the evaluation/deployment module is operatively linked between the research platform module and the production module.

In one embodiment, the data feedback loop further comprises a data ingestion module that ingests data, wherein the data ingestion module is operatively linked between the external feedback/data collection module and the research platform module.

In various embodiments, the research platform module comprises: i) processing input data, and ii) changing weights of the features of a classification model architecture of an already-deployed champion classification model, where the champion classification model has the same feature architecture of a challenger classification model being trained by the research platform module.

In various embodiments, the research platform module comprises: i) processing input data, and ii) changing both features and weights of the features of a classification model architecture of an already-deployed champion classification model.

In another aspect, provided herein is non-transitory computer-readable medium comprising machine-executable code that, upon execution by one or more computer processors, implements a method for creating a diagnostic classifier, the method comprising:
a) obtaining molecular or clinical data from an individual sample associated with a presence or absence of a characteristic of a disease or disorder requiring classification;
b) processing the molecular or clinical data using a data feedback loop system comprising:
   i) a research platform module that trains or re-trains a diagnostic classifier;
   ii) a production module that produces input data, wherein the production module comprises the diagnostic classifier; and
   iii) an external feedback/data collection module that receives data from real-world execution of the diagnostic classifier; and
c) training a diagnostic classifier to provide one or more classification metrics capable of classifying a population of individuals for the disease or disorder.

In one embodiment, the diagnostic classifier comprises a machine learning classifier.

In some examples, the diagnostic classifier is trained using a federated learning approach.

In some examples, the diagnostic classifier is trained using an active learning approach.

In some examples, the machine learning classifier comprises a cancer risk stratification model classifier.

In some examples, the machine learning classifier comprises a lung, breast, prostate, pancreatic, liver, or colorectal cancer risk stratification model classifier.

In some examples, the machine learning classifier comprises a colorectal cancer risk stratification model classifier.

In various embodiments, the training comprises: i) processing input data, and ii) changing weights of the features of a classification model architecture of an already-deployed champion classification model, where the champion classification model has the same feature architecture of a challenger classification model being trained by the research platform module.

In various embodiments, the training comprises: i) processing input data, and ii) changing both features and weights of the features of a classification model architecture of an already-deployed champion classification model.

In one embodiment, the data feedback loop comprises an evaluation/deployment module that productionizes a validated model to prepare for deployment, wherein the evaluation/deployment module is operatively linked between the research platform module and the production module.

In one embodiment, the data feedback loop comprises a data ingestion module that ingests data, wherein the data ingestion module is operatively linked between the external feedback/data collection module and the research platform module.

In another aspect, provided herein is a system comprising a computing device comprising a computer processor, an operating system configured to perform executable instructions, a memory, and a computer program comprising instructions executable by the computer processor to provide a computer application for creating a diagnostic classifier, the instructions comprising:
a) obtaining molecular or clinical data from an individual sample associated with a presence or absence of a characteristic of a disease or disorder requiring classification;
b) processing the molecular or clinical data using a data feedback loop system, wherein the data feedback loop system comprises:
   i) a research platform module that trains or re-trains a diagnostic classifier;
   ii) a production module that produces input data, wherein the production module comprises the diagnostic classifier; and
   iii) an external feedback/data collection module that receives data from real-world execution of the diagnostic classifier; and
c) training a diagnostic classifier to provide one or more classification metrics capable of classifying a population of individuals for the disease or disorder.

In various embodiments, the training comprises: i) processing input data, and ii) changing weights of the features of a classification model architecture of an already-deployed champion classification model, where the champion classification model has the same feature architecture of a challenger classification model being trained by the research platform module.

In various embodiments, the training comprises: i) processing input data, and ii) changing both features and weights of the features of a classification model architecture of an already-deployed champion classification model.

In one embodiment, the classifier model comprises a machine learning classifier.

In some examples, the machine learning classifier comprises a cancer risk stratification model classifier.

In some examples, the machine learning classifier comprises a lung, breast, prostate, pancreatic, liver, or colorectal cancer risk stratification model classifier.

In some examples, the machine learning classifier comprises a colorectal cancer risk stratification model classifier.

In some examples, the machine learning classifier is trained using a federated learning approach.

In some examples, the machine learning classifier is trained using an active learning approach.

In one embodiment, the data feedback loop comprises an evaluation/deployment module that productionizes a validated model to prepare for deployment, wherein the evaluation/deployment module is operatively linked between the research platform module and the production module.

In one embodiment, the data feedback loop comprises a data ingestion module that ingests data, wherein the data ingestion module is operatively linked between the external feedback/data collection module and the research platform module.

In another aspect, provided herein is a system comprising a computing device comprising a computer processor, an operating system configured to perform executable instructions, a memory, and a computer program comprising instructions executable by the computer processor to provide a computer application for re-training a diagnostic classifier, the instructions comprising:
a) obtaining molecular or clinical data from an individual sample associated with a presence or absence of a characteristic of a disease or disorder requiring classification;
b) processing the molecular or clinical data from the individual using a data feedback loop system comprising:
   i) a research platform module that trains or re-trains a diagnostic classifier;
   ii) a production module that produces input data, wherein the production module comprises the diagnostic classifier; and
   iii) an external feedback/data collection module that receives data from real-world execution of the diagnostic classifier; and
a) re-training the diagnostic classifier to improve one or more classification metrics of the diagnostic classifier.

In various embodiments, the training comprises: i) processing input data, and ii) changing weights of the features of a classification model architecture of an already-deployed champion classification model, wherein the champion classification model has the same feature architecture of a challenger classification model being trained by the research platform module.

In various embodiments, the training comprises: i) processing input data, and ii) changing both features and weights of the features of a classification model architecture of an already-deployed champion classification model.

In one embodiment, the classifier model comprises a machine learning classifier.

In some examples, the machine learning classifier comprises a cancer risk stratification model classifier.

In some examples, the machine learning classifier comprises a lung, breast, prostate, pancreatic, liver, or colorectal cancer risk stratification model classifier.

In some examples, the machine learning classifier comprises a colorectal cancer risk stratification model classifier.

In some examples, the machine learning classifier is trained using a federated learning approach.

In some examples, the machine learning classifier is trained using an active learning approach.

In one embodiment, the data feedback loop comprises an evaluation/deployment module that productionizes a validated model to prepare for deployment, wherein the evaluation/deployment module is operatively linked between the research platform module and the production module.

In one embodiment, the data feedback loop comprises a data ingestion module that ingests data, wherein the data ingestion module is operatively linked between the external feedback/data collection module and the research platform module.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Examples of the present disclosure will now be described, by way of example only, with reference to the attached Figures. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A and FIG. 1B provide general and specific schematics of an example feedback loop.

FIG. 2 provides a schematic of an example Research Platform Module useful for a feedback loop.

FIG. 3 provides a schematic of an example Evaluation Environment Module useful for a feedback loop.

FIG. 4 provides a schematic of an example Production Inference Module and External Feedback and Data Collection Module useful for a feedback loop.

FIG. 5 provides a schematic of an example Research Platform Module useful for a feedback loop.

FIG. 6 provides a schematic of a computer system that is programmed or otherwise configured with the machine learning models and classifiers in order to implement methods provided herein.

FIG. 7 provides a schematic of an exemplary federated learning system for a federated learning approach. A federated learning approach is desirable for various embodiments such as, for example, managing potential data privacy or other confidentiality concerns pertaining to sharing of data between organizations.

FIG. 8 provides a schematic of a system for active learning to augment automated generation of outcome labels with human manual labeling in the an efficient manner. This process may involve: 1) a large collection of imperfectly labeled (e.g., un-labeled, partially-labeled, or automatic-labeled only datasets, e.g., labeled only by the automated model, e.g., by natural language processing (NLP) models); 2) an active learner module that selects the automatically-labeled datasets that are most likely to be contributing to model uncertainty; 3) the active learner module that iteratively sends data to the ‘oracle’ (e.g., manual annotation by healthcare professional) for accurate labeling; 4_ an optimal blend of fully manually labeled, and still automatically labeled (or unlabeled) datasets that are sent to train a machine learning module; and 5) trained parameters that are used to build a final classifier.

FIG. 9 provides an exemplary schematic of an example feedback loop.

FIG. 10 provides a schematic showing the operational connection of Evaluation Module, Production Module, Real-world Data and External Data Ingestion Modules useful for a feedback loop.

FIG. 11 provides a schematic showing the operational connection of Production Module with Data Ingestion Modules which comprises Real-world Data and External Data Collection Modules useful for a feedback loop.

FIG. 12 provides a schematic showing the operational connection of Data Ingestion Modules which comprises Real-world Data and External Data Collection Modules useful for a feedback loop.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

As used herein, a recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

As used herein, the term “area under the curve” or “AUC” generally refers to the area under the curve of a receiver operating characteristic (ROC) curve. AUC measures are useful for comparing the accuracy of a classifier across the complete data range. Classifiers with a greater AUC have a greater capacity to classify unknowns correctly between two groups of interest (e.g., cancer samples and normal or control samples). ROC curves are useful for plotting the performance of a particular feature (e.g., any of the biomarkers described herein and/or any item of additional biomedical information) in distinguishing between two populations (e.g., individuals responding and not responding to a therapeutic agent).

As used herein, the term “biological sample” (or just “sample”) generally refers to any substance obtained from a subject. A sample may contain or be presumed to contain analytes for example those described herein (nucleic acids, polyamino acids, carbohydrates, or metabolites) from a subject. In some aspects, a sample can comprise cells and/or cell-free material obtained in vivo, cultured in vitro, or processed in situ, as well as lineages such as pedigree and phylogeny. In various aspects, the biological sample can be tissue (e.g., solid tissue or liquid tissue), such as normal or healthy tissue from the subject. Examples of solid tissue include a primary tumor, a metastasis tumor, a polyp, or an adenoma. Examples of a liquid sample (e.g., a bodily fluid) include whole blood, buffy coat from blood (which can include lymphocytes), urine, saliva, cerebrospinal fluid, plasma, serum, ascites, sputum, sweat, tears, buccal sample, cavity rinse, or organ rinse. In some cases, the liquid is a cell-free liquid that is an essentially cell-free liquid sample or comprises cell-free nucleic acid, e.g., cell-free DNA in some cases, cells, such as circulating tumor cells, can be enriched for or isolated from the liquid.

As used herein, the terms “cancer” and “cancerous” generally refer to or describe the physiological condition in mammals that may be characterized by unregulated cell growth. Neoplasia, malignancy, cancer, and tumor are often used interchangeably and refer to abnormal growth of a tissue or cells that results from excessive cell division.

As used herein, the term “cancer-free” generally refers to a subject who has not been diagnosed with a cancer of that organ or does not have detectable cancer.

As used herein, the term “de-identified data” generally refers to data from which medical information elements such as data and tags that may reasonably be used to identify the patient have been removed (such as, for example, the patient's name, address, social security number, date of birth, contact information). As used herein, the “de-identification” of patient information refers to the removal of at least one of the following individual identifying information characteristics such as names; all geographic subdivisions smaller than a state, such as street address, city, county, precinct, ZIP code (postal code), and equivalent geocodes, except for the initial three digits of the ZIP code (if, according to the current publicly available data from the Bureau of the Census: (1) The geographic unit formed by combining all ZIP codes with the same three initial digits contains more than 20,000 people; and (2) The initial three digits of a ZIP code for all such geographic units containing 20,000 or fewer people is changed to 000); All elements of dates (except year) for dates that are directly related to an individual; telephone numbers; vehicle identifiers and serial numbers, such as license plate numbers; fax numbers; device identifiers and serial numbers; e-mail addresses; Web Universal Resource Locators (URLs); social security numbers; Internet Protocol (IP) addresses; medical record numbers; biometric identifiers, such as fingerprints and voiceprints; health plan beneficiary numbers; full-face photographs and any comparable images; account numbers; any other unique identifying number, characteristic, or code; and certificate/license numbers.

The de-identified data may be counterparts of the original data, produced by “sanitizing” sensitive information within the original data. Once the data is de-identified, the de-identified data may be used for a variety of purposes as described herein, such as research, clinical trials, and so forth, without risking nefarious parties being able to identify individual subjects based on the de-identified data.

As used herein, the term “input features” (or “features”) generally refers to variables that are used by the model to predict an output classification (label) of a sample, e.g., a condition, sequence content (e.g., mutations), suggested data collection operations, or suggested treatments. Values of the variables can be determined for a sample and used to determine a classification. Non-limiting example input features of genetic data include aligned variables that relate to alignment of sequence data (e.g., sequence reads) to a genome and non-aligned variables, e.g., that relate to the sequence content of a sequence read, a measurement of protein or autoantibody, or the mean methylation level at a genomic region.

As used herein, the term “machine learning model” (or “model”) generally refers to a collection of parameters and functions, where the parameters are trained on a set of training samples. The parameters and functions may be a collection of linear algebra operations, non-linear algebra operations, and tensor algebra operations. The parameters and functions may comprise statistical functions, tests, and probability models. The training samples can correspond to samples having measured properties of the sample (e.g., genomic data and other subject data, such as images or health records), as well as observed classifications/labels (e.g., phenotypes or treatments) for the subject. The model can learn from the training samples in a training process that optimizes the parameters (and potentially the functions) to provide an optimal quality metric (e.g., accuracy) for classifying new samples. The training function can comprise expectation maximization, maximum likelihood, Bayesian parameter estimation methods, such as Markov chain Monte Carlo (MCMC), Gibbs sampling, Hamiltonian Monte Carlo (HMC), and variational inference, or gradient based methods such as stochastic gradient descent and the Broyden-Fletcher-Goldfarb-Shanno (BFGS) algorithm. Example parameters include weights (e.g., vector or matrix transformations) that multiply values, e.g., in regression or neural networks, families of probability distributions, or a loss, cost, or objective function that assigns scores and guides model training. Example parameters include weights that multiple values, e.g., in regression or neural networks. A model can comprise multiple submodels, which may be different layers of a model or independent model, each of which may have a different structural form, e.g., a combination of a neural network and a support vector machine (SVM). Non-limiting examples of machine learning models include deep learning models, neural networks (e.g., deep learning neural networks), kernel-based regressions, adaptive basis regression or classification, Bayesian methods, ensemble methods, logistic regression and extensions, Gaussian processes, support vector machines (SVMs), a probabilistic model, and a probabilistic graphical model. A machine learning model can further comprise feature engineering (e.g., gathering of features into a data structure, such as a 1-dimensional, 2-dimensional, or greater dimensional vector) and feature representation (e.g., processing of data structure of features into transformed features to use in training for inference of a classification).

As used herein, the term “subject” generally refers to a mammal such as a human that can be male or female. Such a human can be of various ages, e.g., from 1 day to about 1 year old, about 1 year old to about 3 years old, about 3 years old to about 12 years old, about 13 years old to about 19 years old, about 20 years old to about 40 years old, about 40 years old to about 65 years old, or over 65 years old. In various examples, a subject can be healthy or normal, abnormal, or diagnosed or suspected of being at a risk for a disease. In various examples, a disease comprises a proliferative cell disorder (such as, for example, cancer), a disorder, a symptom, a syndrome, or any combination thereof. As used herein, the terms “subject”, “individual”, or “patient” may be used interchangeably.

As used herein, the term “training sample” generally refers to samples for which a classification may be known. Training samples can be used to train the model. The values of the features for a sample can form an input vector, e.g., a training vector for a training sample. Each element of a training vector (or other input vector) can correspond to a feature that comprises one or more variables. For example, an element of a training vector can correspond to a matrix. The value of the label of a sample can form a vector that contains strings, numbers, bytecode, or any collection of the aforementioned datatypes in any size, dimension, or combination.

As used herein, the terms “tumor”, “neoplasia”, “malignancy”, “proliferative disorder”, or “cancer” generally refer to neoplastic cell growth and proliferation, whether malignant or benign, and all pre-cancerous and cancerous cells and tissues and the result of abnormal and uncontrolled growth of cells.

### I. Diagnostic Feedback Loop

The feedback loops described herein provide an integrated mechanism to generate or improve machine learning classification models by using biological sample molecular data, clinical data, controlled experimental data and real-world clinical data, or a combination thereof.

In one embodiment, a combination of biological sample molecular data, clinical data, controlled experimental data and real-world clinical data are processed using the feedback loop, and a machine learning classifier model is generated or revised as an output of the feedback loop.

In one embodiment, a combination of clinical data, controlled experimental data and real-world clinical data are processed using the feedback loop, and a machine learning classifier is generated or revised as an output of the feedback loop.

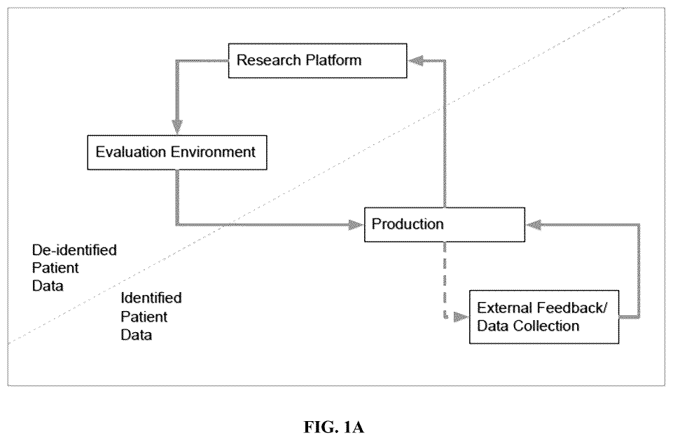

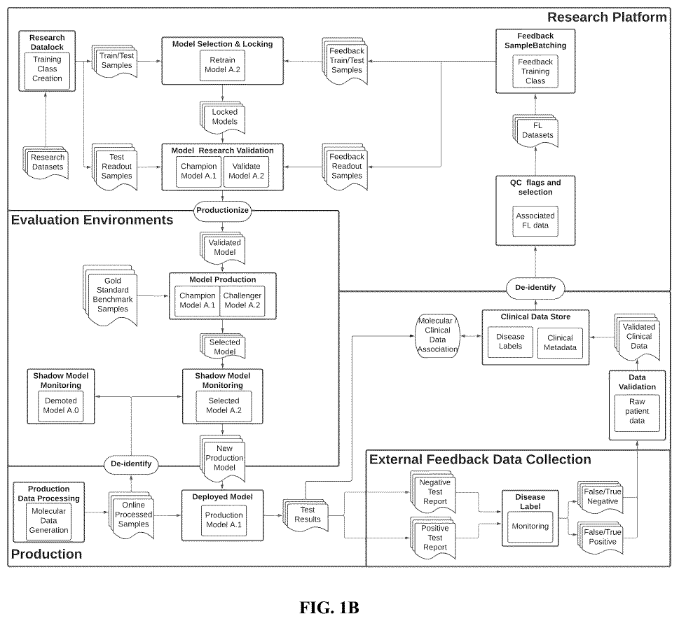

Such a feedback loop may be described, as shown in FIG. 1A comprising modules that in operative communication perform the desired function of the feedback loop. The Research Platform Module may comprise a software environment where research is conducted (e.g., models created, studies run) and where no protected health information (PHI) data that violates The Health Insurance Portability and Accountability Act of 1996 (HIPAA) is allowed (e.g., all clinical data must be de-identified). The Evaluation Environment Module may comprise elements built specifically to evaluate models prior to deployment, and may or may not be a physically different “software environment” than the Research Platform Module. In certain embodiments, activities of the Evaluation Environment Module comprise running two production-grade models head-to-head against each other (e.g., “best” model from research” vs. current model in production) against a selection of sample data. In this Evaluation Environment Module, monitoring may be performed on models in production, e.g., “shadow monitoring,” in which models receive live, de-identified data to generate results. This module may generate parallel predictions for a given sample because multiple models are being run in parallel. Further, the Evaluation Environment Module may provide statistics and quality information on past and candidate models that may be deployed to the final production environment. Procedures and processes may also be modified based on algorithm change protocol/software pre-specifications (ACP/SPS) rules generated by the FDA. The Production Module may comprise a software environment in which real-time patient samples and data are sent and processed. In certain embodiments, this module may comprise controls and elements to manage patient health information that does not need to be de-identified. In various embodiments, the External Feedback/Data Collection Module may refer to process, software, and partnerships that monitor patients in the real-world and sends patient data back to the Production Module.

An example of a more detailed feedback loop is shown in FIG. 1B. In the Production Module, the production data processing element may comprise processing software pipelines that refine molecular data (e.g., data from assays) and outputs BAM files, processed protein data, or other processed sample-level molecular data inputs that have not been featurized. This production data processing element may store molecular data generated in production databases, which may be the raw inputs for generating a prediction and may later be ingested into the research platform. The online processed samples element may receive molecular data that has been processed through the production data processing pipeline. In certain embodiments, whether the patient sample actually has cancer is unknown or unobserved (e.g., the processed samples element is online rather than “offline”, where a cancer label is known or observed). The deployed model element may comprise the model pipeline that refers to the featurization and classifier methods used to receive online samples and generate a prediction. Within the deployed model element, the production model A.1 may comprise a special set of features, weights, parameters, and classifier architecture used to generate a prediction. This model may be verified, validated, and have undergone significant testing to ensure the model is ready for patient use.

In one embodiment, nomenclature for production model “A.1” may refer to a specific, arbitrary model type “A”, and that this is the first version of the model. The model version (or type) may be changed as part of the feedback loop. As such, the nomenclature may refer to iterations of the model and the next iteration may be referred to as “A.2”. The test results element may leverage the result of the production model A.1 to generate a report, which may be sent to the primary care physician (PCP) that ordered the test. In certain embodiments, the Test Results are processed using a molecular/clinical data association element directly, and thus, circumvent the external feedback data collection module. This embodiment may permit refinement of a deployed model with molecular and clinical test data even in the absence of associated disease label input data.

In the data validation (raw patient data) element, the pipeline may be created to connect the negatives/positive disease labels, along with clinical data of interest from the hospital systems to the production environment. Data may undergo quality control checking to ensure that the data meets predesignated specifications. The clinical data store element may comprise a database, where all clinical data bundles are stored as order data or clinical data. The clinical data store element may be where disease labels are also sent. Data may comprise clinical metadata, disease labels, molecular clinical metadata, or a combination thereof. In certain embodiments, clinical metadata may refer to data that is not the disease label sent from a hospital system. In certain embodiments, clinical metadata may comprise order data, as well as additional clinical data fields sent over by a clinical center. In certain embodiments, molecular clinical data association may refer to how clinical data bundles (e.g., order data, disease labels, etc.). Molecular data (e.g., BAMs) may be linked prior to being processed using the research platform module, which allows collected patient data to be queryable and associated in order to be useful in retraining and further investigation.

In the External Feedback Data Collection Module, the negative/positive test report may indicate the likelihood that the patient does or does not have cancer (negative or positive). As a result of this test report, a healthcare professional may recommend follow-up diagnostics (or no follow-up diagnostics in the event of a negative test report). The disease label element may refer to the ground truth “label” that is generated by an established diagnostic pathway.

As an illustrative non-limiting example for colorectal cancer, for example, a colonoscopy report may generate a label of whether a patient does or does not have cancer (the label); until a colonoscopy is generated, whether a patient actually does or does not have cancer may not be known or observed regardless of what was predicted by the CRC test.

Further, molecular data obtained from a patient biological sample may lack a label associated with a disease, disorder, or symptom until a clinical report is entered into an electronic medical record (EMR) and matched with the patient molecular data in the External Feedback/Data Collection Module.

Similar reports for diagnostic tests for other cancer types may be managed in a similar manner. These disease labels may be required to retrain the model. Within the disease label element, the monitoring may comprise the processes, partnerships, and pipelines to collect disease labels and other clinical data of interest for retraining and generating new models. One output of the disease label element may comprise false/true negative end test report, and the likely course of action/type of data outputted to the data validation element. In the event that a patient receives a negative test report, a false negative means that the patient does, in fact, have cancer. As a negative test report may likely not lead up to a diagnostic follow-up, such as a colonoscopy if referring to CRC test, false/true negatives may likely not be sent back to the production module and ingested into the feedback loop. Another output of the disease label element may comprise a false/true positive end test report, and the likely course of action/type of data outputted to the data validation element. In some embodiments, false/true positives may indicate that a patient received a diagnostic test, such as a colonoscopy if referring to CRC test.

In one embodiment, incoming data may be received in the research platform after being de-identified, which removes PHI fields to ensure HIPAA compliance. The quality control (QC) flags and selection element may comprise QC procedures to ensure that ingested RWD meets internal standards for quality and is in a form usable in the models contained in the feedback loop. Within the QC flags and selection element, the Associated Feedback Loop (FL) data may refer to FL data (such as molecular and clinical data) that can be associated with a patient and used within the research platform. The FL Datasets may comprise a collection of molecular data that has been processed through assays in production, which can be featurized and then processed using a model retraining process. In certain embodiments, data are obtained from real-world patients, and are separate from molecular data collections derived from structure clinical trial collection studies. The feedback sample batching element may comprise a process for generating a list of datasets that has undergone QC review and scientific bar for usage in retraining and evaluation, for RWD input.

The feedback training classes may be the “list” of datasets that can be used for retraining and model validation. These training classes may be all from RWD sources, and the molecular data may be generated from a production pipeline. The research datalock element may comprise a process for generating a list of datasets that has undergone QC and scientific bar for usage in retraining and evaluation generated from studies that is not for RWD input. This process may provide traceability and ensure that only datasets that have met the bar for quality are used in the process.

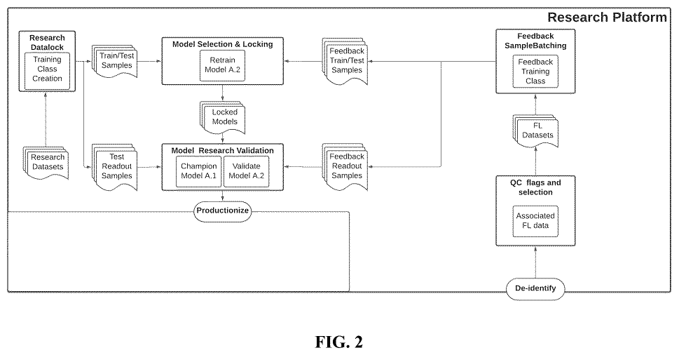

Research datasets may be molecular samples that have run through the entire research platform module (FIG. 2) and may be used as raw inputs for a featurization pipeline. In certain embodiments, these datasets are generated from clinical studies in conventional collection methods. Within the research datalock, the training class creation may comprise a list of datasets that has cleared QC and scientific bar for usage in retraining and evaluation generated from studies not for RWD input.

The model selection and locking element may comprise a process for retraining an existing model spec and generating new hyperparameters. This element may receive training classes as an input and mix feedback loop and normal training classes for retraining an existing model spec. The feedback loop training classes may “boost” the number of total samples used for training a model. Within the model selection and locking element, the retrain model A.2 may comprise a model that is generated from the new “mixture” of training classes and feedback loop training classes that are processed using the loop. This element may use an existing model specification, so the version is changed from model version A.1 to model version A.2. This process may leverage a model architecture that is already used in production. Locked models may comprise models that has been retrained as part of the “Model Selection and Locking” operation and ready for validation, which are processed using the model research validation element. The model research validation element may comprise a process for generating unbiased performance estimates from samples that the model has not been trained on. The feedback readout samples may comprise data training classes from the feedback loop (using RWD) used specifically to evaluate the new model as part of a readout holdout dataset. Test readout samples may comprise data training classes used specifically to evaluate the new model as part of a holdout readout that are not generated from RWD and compare against a model currently in the Production Module. Within the model research validation element, the Champion Model A.1 may refer to the best performing model that is currently in production within the Production Module. The active model may be evaluated against incoming data received from the Production Module. The validated model A.2 may refer to the new candidate model (also a “challenger” model) that has been retrained using incoming data and is being tested on new data such as, for example, a holdout data set.

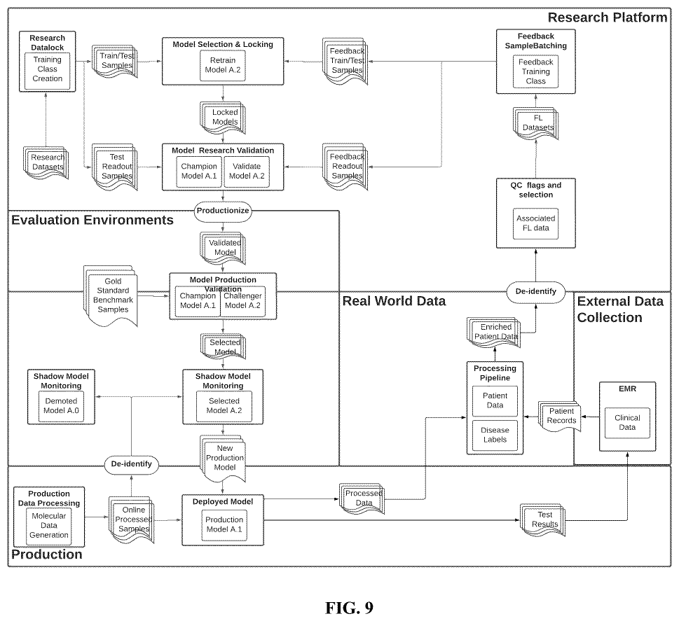

Another feedback loop schema is shown in FIG. 9. In the Production Module, the Production Data Processing element may comprise processing software pipelines that refine molecular data (e.g., data from assays) and outputs BAM files, processed protein data, or other processed sample-level molecular data inputs that have not been featurized. This element may store molecular data generated in production databases, which may be the raw inputs for generating a prediction and may later be ingested into the research platform. The Online Processed Samples element may receive molecular data that has been processed through the Production Data Processing pipeline. In certain embodiments, whether the patient sample actually has a disease such as cancer is unknown or unobserved (e.g., the Processed Samples element is online rather than “offline”, where a cancer label is known or observed). The Deployed Model element may comprise the model pipeline that refers to the featurization and classifier methods used to take in online samples and generate a prediction. Within the Deployed Model element, the Production Model A.1 may comprise a special set of features, weights, parameters, and classifier architecture used to generate a prediction. This model may be verified, validated, and have undergone significant testing to ensure the model is ready for patient use. Production Model “A.1” may refer to a specific, arbitrary model type “A”, and that this is the first version of the model. The model version (or type) may be changed as part of the feedback loop. As such, the nomenclature may refer to iterations of the model and the next iteration may be referred to as “A.2”. The Test Results element may leverage the result of the production model A.1 to generate a report, which may be sent to the PCP that ordered the test and become part of the EMR for a patient as shown in the External Data Collection Module. The Processed Data from the Deployed Model element proceeds to the Real-world Data Module (RWD Module).

The RWD Module provides an optional ingestion pathways from the Product Pipeline, providing a secure location for PHI data outside of the Production Environment Module. This data may then be curated, integrated with other RWD production streams, and de-identified to be safely processed and used in the Research Platform Module to preserve data security and patient privacy. In some embodiments, Patient Data is processed using the Research Platform Module in a form that is curated, reliable, useful for research efforts, and meets regulatory standards (HIPAA, system integration timelines, etc.) as part of the feedback loop and RWD ingestion and use. In the RWD Module, Processed Data from the Production Module and Patient Record Information from the External Data Collection Module may be received by the RWD Processing Pipeline element. The RWD Processing Pipeline may output Enriched Patient Data, which is then de-identified as described herein before being processed using a Research Platform Module described herein.

In the External Feedback Data Collection Module, the Negative/Positive Test Report may indicate the likelihood that the patient does or does not have cancer (negative or positive). As a result of this test report, a healthcare professional may recommend follow-up diagnostics (or no follow-up diagnostics in the event of a negative test report). The Disease Label element may refer to the ground truth “label” that is generated by an established diagnostic pathway.

In one embodiment, for colorectal cancer, for example, a colonoscopy report may generate a label of whether this patient does or does not have cancer (the label); until a colonoscopy is generated, whether a patient actually does or does not have cancer may not be known or observed regardless of what was predicted by the CRC test. Similar reports for diagnostic tests for other cancer types may be managed in a similar manner. These disease labels are required to retrain the model. Within the Disease Label element, the Monitoring may comprise the processes, partnerships, and pipelines to collect disease labels and other clinical data of interest for retraining and generating new models. One output of the Disease Label element may comprise False/True Negative end test report, and the likely course of action/type of data outputted to the Data Validation element. In the event that a patient receives a negative test report, a false negative means that the patient does in fact have cancer. As a negative test report may likely not lead up to a diagnostic follow-up, such as a colonoscopy if referring to CRC test, false/true negatives may likely not be sent back to the production system/ingested into the feedback loop. Another output of the Disease Label element may comprise a False/True Positive end test report, and the likely course of action/type of data outputted to the Data Validation element. False/True positives may indicate that a patient received a diagnostic test, such as a colonoscopy if referring to CRC test. Test Results are received in the External Data Collection Module and become part of the EMR element of the Patient Records data. The Patient Records data is received by the Processing Pipeline element in the RWD Module.

In the Research Platform, incoming data from the RWD Module may be received after being de-identified, which removes PHI fields to ensure HIPAA compliance. The QC flags and selection element may comprise QC procedures to ensure that processed (or ingested) RWD meets internal standards for quality and is in a form usable in the models contained in the feedback loop. Within the QC flags and selection element, the Associated FL data may refer to Feedback Loop data (such as molecular and clinical data) that can be associated with a patient and used within the research platform. The FL Datasets may comprise a collection of molecular data that has been processed through assays in production, which can be featurized and then processed using a model retraining process. In certain embodiments, data are obtained from real-world patients, and are separate from molecular data collections derived from structure clinical trial collection studies. The Feedback Sample Batching element may comprise a process for generating a list of datasets that has undergone QC review and scientific bar for usage in retraining and evaluation, for RWD input.

The Feedback Training Classes may be the “list” of datasets that can be used for retraining and model validation. These training classes may be all from RWD sources, and the molecular data may be generated from a production pipeline. The Research Datalock element may comprise a process for generating a list of datasets that has undergone QC and scientific bar for usage in retraining and evaluation generated from studies that is not for RWD input. This process may provide traceability and ensure that only datasets that have met the bar for quality are used in the process.

Research Datasets may be molecular samples that have run through the entire Research Platform module and may be used as raw inputs for a featurization pipeline. In certain embodiments, these datasets are generated from clinical studies in conventional collection methods. Within the Research Datalock, the Training Class Creation may comprise a list of datasets that has cleared QC and scientific bar for usage in retraining and evaluation generated from studies not for RWD input.

The Model Selection and Locking element may comprise a process for retraining an existing model specification and generating new hyperparameters. This element may receive training classes as an input and mix feedback loop and normal training classes for retraining an existing model spec. The feedback loop training classes may “boost” the number of total samples used for training a model. Within the Model Selection and Locking element, the Retrain Model A.2 may comprise a model that is generated from the new “mixture” of training classes and feedback loop training classes that are processed using the loop. This element may use an existing model specification, so the version is changed from model version A.1 to model version A.2. This process may leverage a model architecture that is used already in production. Locked Models may comprise models that has been retrained as part of the “Model Selection and Locking” operation and ready for validation, which are processed using the Model Research Validation element. The Model Research Validation element may comprise a process for generating unbiased performance estimates from samples that the model has not been trained on. The Feedback Readout Samples may comprise data training classes from the feedback loop (using RWD) used specifically to evaluate the new model as part of a readout holdout dataset. Test Readout Samples may comprise data training classes used specifically to evaluate the new model as part of a holdout readout that are not generated from RWD and compare against a model currently in the Production Module. Within the Model Research Validation element, the Champion Model A.1 may refer to the best performing model that is currently in production within the Production Module. The active model may be evaluated against incoming data received from the Production Module. The Validate Model A.2 may refer to the new candidate model that has been retrained using incoming data and is being tested on new, holdout data.

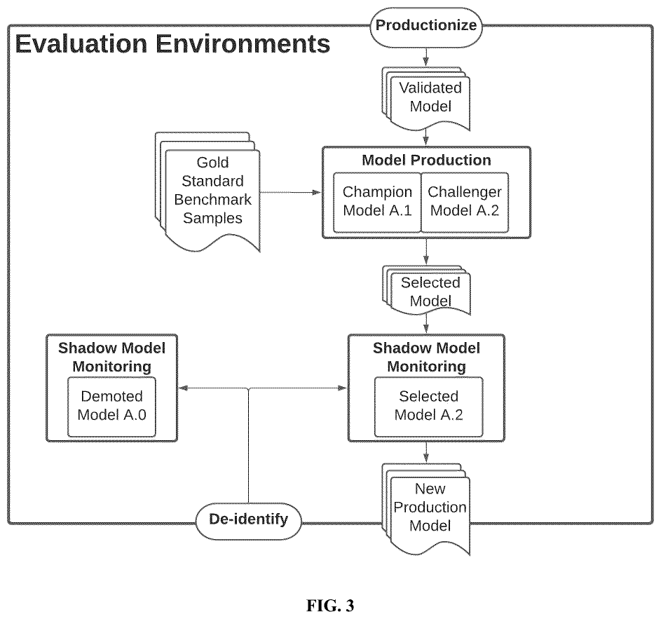

From the Research Platform, a model may flow through a Productionize process that may comprise a process of refining research code and transferring a selected model to a new codebase that is acceptable for the production environment (FIG. 3). If the Validate Model A.2 performs better than Champion Model A.1, then the Validate Model A.2 may flow through “productionize” into the Evaluation Environment Module.

In the Evaluation Environment Module, the Validated Model may comprise the new model that has been validated and productionized from the Research Platform (as used herein a “challenger model”). The Model Production element may comprise a process for comparing performance of models that are all production-grade code in a head-to-head comparison. The Gold Standard Benchmark Samples may comprise a set of samples used specifically to provide a readout of the final head-to-head comparison of the deployed model (as used herein “a champion model”) and the challenger model.

In some embodiments, a final quality control check may be performed before moving to push a model to production environment where the model may be used on live patients.

Within the Model Production element, Champion Model A.1 may refer to the existing model that is currently in production being used on live patients undergoing screening for disease in a clinic. The Challenger Model A.2 may refer to the new model that has just been productionized and retrained. The Selected Model may refer to the Challenger Model A.2 if the performance of the Selected Model exceeds that of Champion Model A.1 using the Gold Standard Benchmark Samples. The Shadow Model Monitoring element may generate predictions using de-identified data across multiple models. This element may be used to de-risk models prior to deployment in the Production module. The Shadow Monitoring element may be used to assess how older models are performing on live patient data obtained in the Production Module. This element may generate predictions on “live data” that does not have labels. The value may be used to identify anomalies and generate long-term performance statistics. Within the Shadow Model Monitoring element, Selected Model A.2 may comprise a model that is being prepared for deployment to the production pipeline in the Production Module. Prior to switching models from A.1 to A.2, the Selected Model A.2 may be assessed using live data for a predetermined period of time to ensure that there are no anomalies and to ensure highest confidence possible in results. Within the Shadow Model Monitoring element, the Demoted Model A.0 may refer to older models that can still be evaluated on live data to assess for any quality problems that become available.

After satisfying preset criteria of the Evaluation Environment module, a Selected Model A.2 may flow through “New Production Model” into the Production Module Deployed Model Element. The New Production Model may comprise the newly deployed production model that has been generated by the feedback loop, productionized, and then validated and is ready for use as the new “Deployed Model” element in the feedback loop. FIG. 9 provides an exemplary schematic of an example feedback loop described herein.

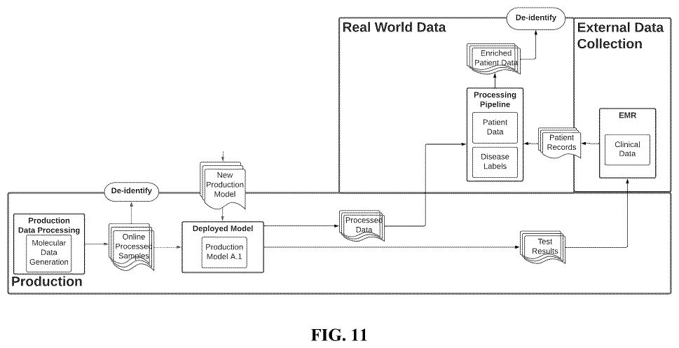

FIG. 11 provides a schematic showing the operational communication of a Production Module with Data Ingestion Modules which comprises RWD and External Data Collection Modules useful for the feedback loops described herein.

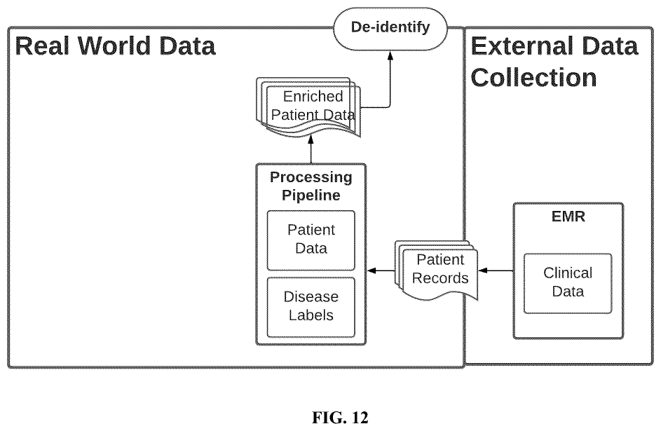

FIG. 12 provides a schematic of Data Ingestion Modules which comprises RWD and External Data Collection Modules useful for the feedback loops described herein.

In one aspect, provided herein is a data feedback loop comprising:
a research platform module that trains or re-trains a classification model;
a production module that produces input data, wherein the production module comprises the classification model; and
an external feedback/data collection module that receives data from real-world execution of the classification model, wherein is the external feedback/data collection module operatively linked to the research platform module.

In one aspect, provided herein is a data feedback loop comprising:
a cohort selection and retraining module that selects classes of training samples for a classification model or re-trains the classification model;
a product inference module that produces raw data for ingestion into the data feedback loop system; and
an external feedback/data collection module that receives data from real-world execution of the classification model.

In one aspect, provided herein is a data feedback loop comprising:
a research platform module that trains or re-trains a classification model; and
a production module produces input data, wherein the production module comprises the classification model.

In one aspect, provided herein is a data feedback loop comprising:
a cohort selection and retraining module that selects classes of training samples for a classification model or re-trains the classification model; and
a product inference module that produces raw data for ingestion into the data feedback loop system.

In one embodiment, the external feedback/data collection module is operatively linked to the cohort selection and retraining module.

In one embodiment, the data feedback loop comprises an evaluation/deployment module that provides productionizing of a validated model to prepare for deployment, and the evaluation/deployment module is operatively linked between the cohort selection and retraining module and the product inference module.

In one embodiment, the data feedback loop comprises a data ingestion module that ingests data, and the data ingestion module is operatively linked between the external feedback/data collection module and the cohort selection and retraining module.

In one embodiment, the data feedback loop comprises directional information flow from the evaluation/deployment module to the product inference module and either back to the evaluation/deployment module or forward to the external feedback/data collection module.

In one embodiment, the data feedback loop comprises a research ingestion module that processes clinical metadata or labels with quality control metrics, matches the clinical metadata with patient molecular data, or pushes the matched clinical metadata and molecular data to the research platform module, and the research ingestion module is operatively linked between the external feedback/data collection module and the cohort selection and retraining module.

In one embodiment, the data feedback loop comprises directional information flow from cohort selection and retraining module to produce inference module to external feedback/data collection module and returning to the cohort selection and retraining module.

In some embodiments, the biological sample obtained from the subject comprises body fluids, stool, colonic effluent, urine, blood plasma, blood serum, whole blood, isolated blood cells, cells isolated from the blood, or a combination thereof.

In some embodiments, the cell proliferative disorder is colorectal, prostate, lung, breast, pancreatic, ovarian, uterine, liver, esophagus, stomach, or thyroid cell proliferation.

In some embodiments, the cell proliferative disorder comprises colon adenocarcinoma, liver hepatocellular carcinoma, lung adenocarcinoma, lung squamous cell carcinoma, ovarian serious cystadenocarcinoma, pancreatic adenocarcinoma, prostate adenocarcinoma, or rectum adenocarcinoma.

In some embodiments, the cell proliferative disorder is stage 1 cancer, stage 2 cancer, stage 3 cancer, stage 4 cancer, or combination of stages.

### A. Research Platform Module

A Research Platform Module may employ pre-selected classes or cohorts of individual or subject data for training a new classification model or re-training an existing classification model located on a registry stored within the Research Platform Module.

In one embodiment, the pre-selected classes or cohorts of patient data contain de-identified individual or subject data where identifiable features are removed from the data before ingestion into the Research Platform Module for subject security purposes. FIG. 2 provides a general schematic of a Research Platform Module that may be useful for the feedback loops described herein.

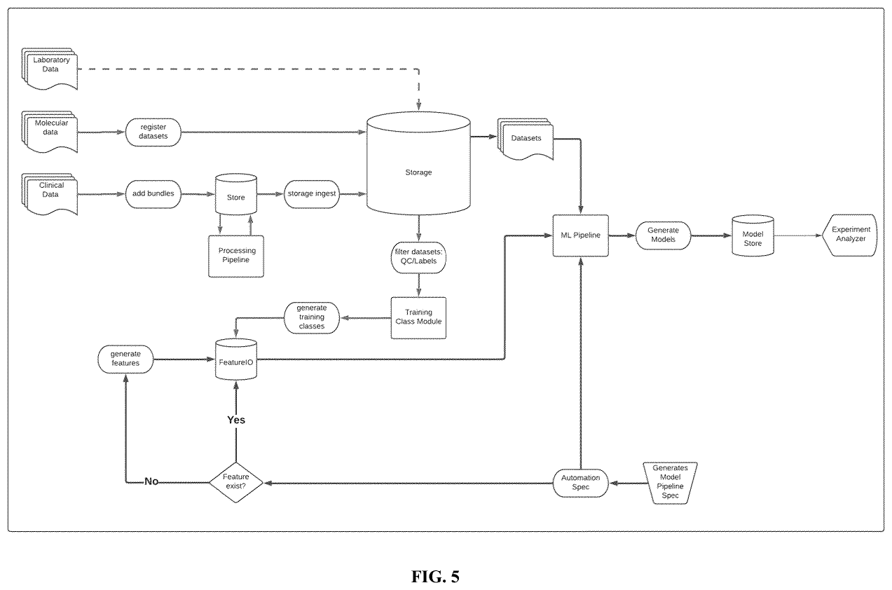

FIG. 5 provides another schematic of a general Research Platform Module isolated from the other modules of the feedback loop that may be useful for the feedback loops described herein.

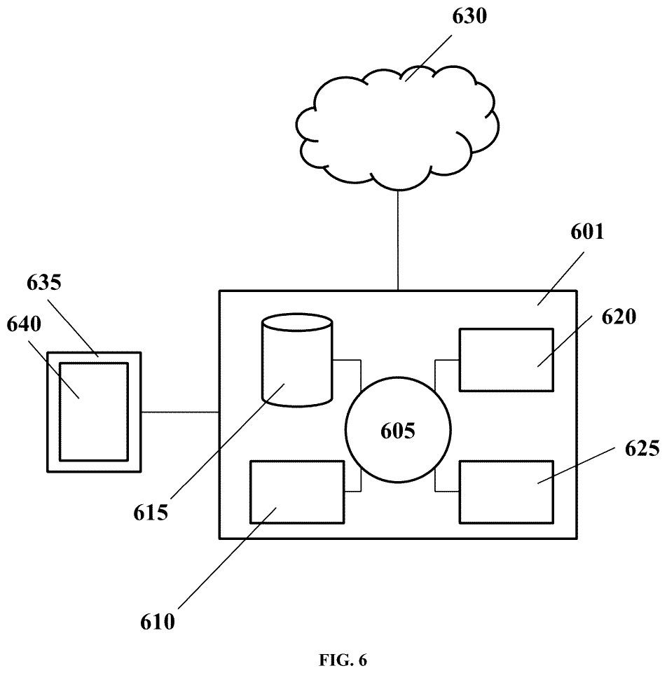

FIG. 6 provides a schematic of a computer system usable in the research platform module that is programmed or otherwise configured with the machine learning models and classifiers in order to implement methods provided herein.

A Research Platform Module may comprise Cohort Selection Training/Retraining modules and may utilize validated de-identified data associated with characteristics of a populations of individuals. This data may be selected to provide classes of training samples and allow for retraining of specified models.

In various embodiments, the Model Selection and Locking element of the Research Module is configured with computational tools to apply machine learning approaches to process input data to design model architectures and/or train model architectures to provide classification models.

In certain embodiments, the computational tools may comprise multilevel medical embedding (MiME), graph convolutional transformer (GCT), deep patient graph convolutional network (DeePaN), convolutional autoencoder network (ConvAE), temporal phenotyping, BEHRT, Med-BERT, GenNet deep learning Framework, among other tools and approaches to generate and train classification models.

In certain embodiments, data embedding is employed in the machine learning approaches. In some embodiments, the data embedding is patient-level embedding.

In one embodiment, the classifier model comprises a machine learning classifier.

In some examples, the machine learning classifier comprises a cancer risk stratification model classifier.

In some examples, the machine learning classifier comprises a lung, breast, prostate, pancreatic, liver, or colorectal cancer risk stratification model classifier.

In some examples, the machine learning classifier comprises a colorectal cancer risk stratification model classifier.

In some examples, the machine learning classifier is trained using a federated learning approach.

In some examples, the machine learning classifier is trained using an active learning approach.

In one embodiment, the data feedback loop comprises an evaluation/deployment module that productionizes a validated model to prepare for deployment, wherein the evaluation/deployment module is operatively linked between the research platform module and the production module.

In one embodiment, the data feedback loop comprises a data ingestion module that ingests data, wherein the data ingestion module is operatively linked between the external feedback/data collection module and the research platform module.

In various embodiments, the classifier model comprises a machine learning model that is portable or deployed in the form of a federated learning approach, to address the growing industry concerns about the potential for transfer of even de-identified patient data (since such data as genomic data, may conceivably become identifiable), and to overcome other potential obstacles with data sharing between health care organizations and particular devices, servers, and EHR systems. A federated learning approach may train a machine learning model by transferring a model in need of training to a location where the data is stored, as opposed to the data being transferred to a server running the machine learning model to be trained, which may be typical in traditional non-federated learning approaches. Optionally, any of the machine learning models disclosed herein may potentially be deployed in the form of a federated learning approach as opposed to the traditional centralized learning approach. Federated learning may be useful in cases in which moving the actual data for training or validation to the machine learning model is either difficult or impossible. In these instances, moving the machine learning model to the data may instead be more realistic and practical. This may be the case due to constraints due to size of data, data transfer challenges, privacy, or contractual and ownership concerns, or a combination of these, leading to challenges moving data from behind the firewall of an institution where the data is stored, or from a device such as a wearable or mobile device.

Federated learning offers an alternative to the traditional default centralized learning approach, in which the data from one or more sources, such as EHR or other data transferred is sent for training to a centralized machine learning model on a central server.

In federated learning, the model is sent to the data, such as in one of several potential formats, for example, federated learning with Application Server aggregation, or federated learning using Peer-to-Peer model transfer. Some versions of federated learning may include one or more local training environments, for instance, a computational environment behind the firewall of a health system (whether cloud, virtual private machine, or on-premises computer capability) with access to locally protected datasets.

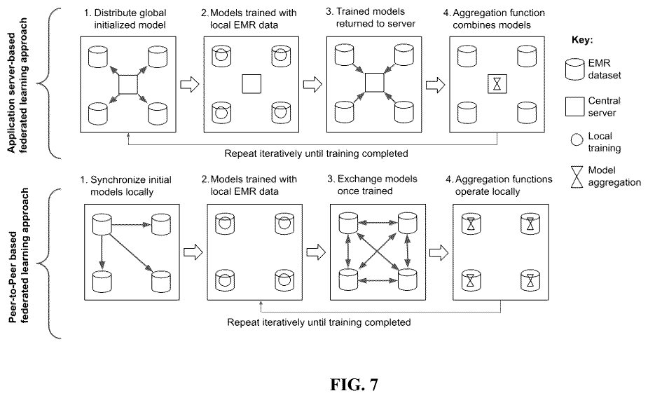

FIG. 7 demonstrates a federated learning approach to model training useful in the feedback loops described herein, with an Application Server based approach (upper), the machine learning model is first instantiated as an Operation 1 (e.g., model is created and parameters are initialized, such as with small random numbers) on a central application server, from where the model is distributed to the local training environments (shown in the four corners, as depicted in FIG. 7). In Operation 2, versions of the model may then be trained locally on those local environments using the local data held therein, using training approaches such as Stochastic Gradient Descent (SGD) or batch learning. In Operation 3, the trained models are returned to the application server. In Operation 4, the models are aggregated using one or more of many possible aggregation functions, such as simple averaging or other suitable aggregation functions. After the aggregation function operation, the whole procedure may be repeated iteratively with a view to improving and refining the trained model by successive training phases, or epochs, in which all of the training data is used again. Similarly, with Peer-to-Peer federated learning (as shown in the lower section of FIG. 7), the machine learning model is instantiated such as in one of the local training environments. Versions of the model are distributed laterally to the other peer local environments (Operation 1). The models may then all be trained in parallel (Operation 2). The models are then exchanged laterally for further training or fine-tuning in the other local training environments (Operation 3). The models are then aggregated, without the need for an application server (Operation 4), because the aggregation occurs locally in the peer-to-peer version. As with the application server-based approach, iterations of the whole peer-to-peer process may then be successively performed to develop and improve the model.

In certain embodiments, the training and validation data classes are generated manually.

In other embodiments, the training and validation data classes are generated automatically.

In other embodiments, the data feedback loop incorporates an active learning approach to attain an optimal balance between generation of training and validation data classes generated manually with those generated automatically.

In these embodiments, a specialized Active Learner machine learning model requests hand-labeling or manual annotation of data from the EHR in the form of manual review by clinician or other health professional. This active learning allows refinement and improvement of the quality of the data, such as health outcome data, and thereby achieves better classification. In one aspect, the machine learning model automatically and iteratively requests which EHR examples to be assessed by manual review in order to minimize uncertainty, based on some learned representation of the EHR or other features of the patient level input data.

Active Learning approaches may be useful to optimize use of a finite resource such as time-consuming and potentially prohibitively expensive process of manual annotation and data extraction from EHR by a healthcare professional, who might take many minutes or hours to extract the data as opposed to fractions of a second for an automated model. Active Learning might be used in cases where there is finite capacity to manually annotate a partial fraction, such as 0.01%, 0.1%, 1%, 10%, or 50% of the whole dataset, for example. Rather than allocating such a finite resource randomly across the dataset, Active Learning may be used to learn to choose which unlabeled or imperfectly labeled data samples may be manually labeled to get the best overall final result in an efficient manner to increase data integrity and usefulness for large datasets to be handled by the feedback loops described herein.

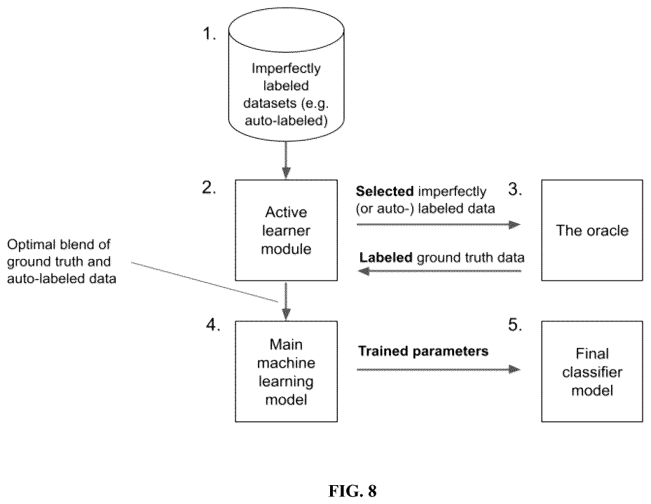

An active learning approach may have a goal of achieving a trained classifier to detect disease, drug responsiveness, prognosis in which large amounts of data are required for accurate model development and training, but input data may be imperfectly labelled. As depicted in FIG. 8, the process starts (Operation 1) with a volume of imperfectly labeled data that includes unlabeled data, poorly-labeled data, or data that has been labeled by an automated annotation process, such as natural language processing or a combination thereof. Automated annotation may be imperfect, with some level of false positive and false negative errors. As such, the labels may be improved towards ground truth using a more labor-intensive process of manual annotation by healthcare professionals able to review the EHR in detail and extract data to a tabulated or tokenized format. In Operation 2, an Active Learner Module selects datasets to send to the “oracle” for manual annotation in Operation 3. As used herein, the term “oracle” refers to the source of ground truth. In this case, the oracle is the manual annotation process by an individual, which is time consuming at large scale. The initial selection of which datasets to send to the oracle may be random or may be set by predetermined rules or heuristics. The oracle then performs the manual labeling process and returns labeled ground truth data to the Active Learner module, such that the amount of perfectly labeled ground truth data increases, but with the highest impact datasets being manually labeled. By way of successive iterative cycles between the Operations 2 and 3, the Active Learner module may learn the predictive characteristics of the datasets that make them most beneficial to be sent to the oracle overall, based on feedback the Active Learner receives, with a goal of minimizing the uncertainty of the Active Learner module, or improving the performance of the downstream classifier.

The partially ground truth labeled dataset is then processed using the main machine learning classifier in Operation 4 for training. The classifier model may be of any type of such classifier model, such as: logistic regression model, support vector machine (SVM), random forest (RF), multi-layer perceptron (MLP), convolutional neural network model (CNN), recurrent neural network (RNN), self-attention or Transformer-based model, or any other suitable classifier model. In Operation 5, the final trained parameters may be passed to the final locked classifier model.

In one embodiment, the system comprises a feedback loop from the final classifier predictions back to the Active Learner to enable end-to-end training based on the prediction accuracy of the classifier. Any of the operations from Operation 5 back to Operation 1 may have feedback loops and be repeated iteratively to improve the final results.

In some embodiments, an active learning module may be implemented using active learning approaches such as those in the various categories of stream-based selective sampling, pool based sampling, or optionally membership query synthesis, and by non-limiting example, may include specific approaches and choices such as Expected Model Change, Expected Error Reduction, Exponentiated Gradient Exploration, Uncertainty Sampling, Query by Committee, Querying from Diverse Subspaces or Partitions, Variance Reduction, Conformal Predictors, Mismatch-First Farthest Traversal, User-Centered Labeling Strategies, and Active Thompson Sampling. In certain embodiments, the methods disclosed herein may also optionally be combined with conceptually related approaches such as reinforcement learning (RL).

In one embodiment, the research platform module provides automatic featurization of input data meeting predetermined specifications and automatic processing of featurized input data into the machine learning pipeline.

In one embodiment, the machine learning pipeline comprises model selection and locking elements and model research validation elements.

In various embodiments, inputs to this module are selected from: de-identified, matched patient data, feedback batching specifications, ingested data QC specifications, and pre-existing classification models.

One element of the Research Platform module may match the clinical metadata with patient molecular data, and may push this matched clinical and molecular data to the Evaluation Environment module.

In various embodiments, patient data comprises biological sample molecular data, clinical data, controlled experimental data and real-world clinical data, or a combination thereof.

In certain embodiments, molecular data comprises information derived from molecules in a biological sample from a subject such as, but not limited to, nucleic acid sequence, length, end point, mid point, methylation status or mutation information; protein sequence, abundance, profile, binding affinity information; autoantibody abundance, profile, diversity information; and metabolite abundance, profile, diversity.

In one embodiment, a combination of clinical data, controlled experimental data and real-world clinical data are processed using the feedback loop, and a machine learning classifier is generated or revised as an output of the feedback loop.

In various embodiments, the Research Platform Module comprises: i) processing input data, and ii) changing weights of the features of a classification model architecture of an already-deployed champion classification model, where the champion classification model has the same feature architecture of a challenger classification model being trained by the Research Platform module.

In various embodiments, the Research Platform Module comprises: i) processing input data, and ii) changing both features and weights of the features of a classification model architecture of an already-deployed champion classification model.

In various embodiments, outputs of this module comprise validated classification models. In certain embodiments, the validated classification model has demonstrated performance in classifying a population of individuals or samples based on preselected characteristics.

In certain embodiments, incoming patient data from the production module is subjected to a quality control analysis and matched with patient labels used in the classification models. In various embodiments, ratios of incoming patient data and data from prior model validation are varied to train and validate classification models in the Research Platform Environment.

In certain embodiments, the training data class comprises approximately 90% prior model data and 10% incoming patient data, approximately 80% prior model data and 20% incoming patient data, approximately 70% prior model data and 30% incoming patient data, approximately 60% prior model data and 40% incoming patient data, approximately 50% prior model data and 50% incoming patient data, or approximately 40% prior model data and 60% incoming patient data.

In certain embodiments, the validation data class comprises approximately 90% prior model data and 10% incoming patient data, approximately 80% prior model data and 20% incoming patient data, approximately 70% prior model data and 30% incoming patient data, approximately 60% prior model data and 40% incoming patient data, approximately 50% prior model data and 50% incoming patient data, or approximately 40% prior model data and 60% incoming patient data.

### B. Evaluation Environment

An Evaluation Environment module similarly may use validated models output from the Research Platform Model and monitor and evaluate these models for deployment.

An Evaluation Environment module may comprise an Evaluation/Deployment module to provide productionizing of a validated model to prepare for deployment. In this module, unbiased performance may be estimated, a validated model may be verified in the Evaluation Environment module, and monitored prior to deployment. In certain embodiments, the Evaluation Environment module also provides shadow model monitoring for a new production model that is deployed as the output of this module.

FIG. 3 provides a schematic of an example of an Evaluation Environment module that may be useful for the feedback loops described herein.

From the research platform, a model may flow through a productionize process that may comprise a process of refining research code and transferring a selected model to a new codebase that is acceptable for the production environment. If the validate model (“challenger”) A.2 performs better than champion model A.1, then the validate model A.2 may flow through the productionize process into the Evaluation Environment module.

In the Evaluation Environment module, the validated model may comprise the new model that has been validated and productionized from the research platform. The model production element may comprise a process for comparing performance of models that are all production-grade code in a head-to-head comparison. The gold standard benchmark samples may comprise a set of samples used specifically to provide a readout of the final head-to-head comparison of the champion and the challenger model. In some embodiments, a final quality control check may be performed before moving to push a model to production environment (e.g., deployed) where the model may be used on live patients. Within the model production element, champion model A.1 may refer to the existing model that is currently in production being used on live patients undergoing screening for disease in a clinic. The challenger model A.2 may refer to the new model that has just been productionized and retrained. The selected model may refer to the challenger model A.2 if the performance of the selected model exceeds that of champion model A.1 using the gold standard benchmark samples. The shadow model monitoring element may generate predictions using de-identified data across multiple models. This element may be used to de-risk models prior to deployment in the Production module. The shadow monitoring element may be used to assess how older models are performing on live patient data obtained in the production module. This element may generate predictions on “live data” that does not have labels. The value may be used to identify anomalies and generate long-term performance statistics. Within the shadow model monitoring element, selected Model A.2 may comprise a model that is being prepared for deployment to the production pipeline in the production module. Prior to switching models from A.1 to A.2, the selected Model A.2 may be assessed using live data for a predetermined period of time to ensure that there are no anomalies and to ensure highest confidence possible in results. Within the shadow model monitoring element, the demoted Model A.0 may refer to older models that can still be evaluated on live data to assess for any quality problems that become available.

After satisfying preset criteria of the evaluation environment module, a selected model A.2 may flow through the productionize process and be deployed into the Production module. The new production model may comprise the newly deployed production model that has been generated by the feedback loop, productionized, and then validated and is ready for patients.

In various embodiments, inputs to this module are selected from: a validated classification model, gold standard data sets (for example clinically controlled and validated data sets), or de-identified molecular data.

In various embodiments, a validated classification model (“a challenger”) is compared to a deployed classification model (“a champion”), which serves as the baseline deployed production model. For a challenger model to usurp the champion model and replace the champion model as the new deployed production model, the challenger model must have improved performance over the champion model in various performance metrics.

In various embodiments, the performance metrics are selected from: 1) accuracy of classification with a learned threshold among positive examples (e.g., true positive rate or, equivalently, sensitivity); 2) accuracy of classification with a learned threshold among negative examples (e.g., true negative rate or, equivalently, specificity); 3) accuracy of classification among positive examples at a calibrated specificity among negative samples; 4) balanced accuracy across both positive and negative examples; 5) area under the receiver operating characteristic curve (AUROC); 6) partial AUROC, restricting to specificity ranges of interest; 7) area under the precision-recall curve (AUPRC); 8) partial AUPRC, restricting to precision ranges of interest; and a combination thereof. In certain embodiments, the aforementioned performance metrics may be evaluated multiple times using Monte Carlo resampling or other perturbation techniques to assess statistical significance of improvement.

In certain embodiments, a challenger model may also be selected over a champion model if the challenger model is (i) non-inferior to the champion model in view of the aforementioned performance metrics; and (ii) provides other benefits, for example, but not limited to, increased robustness to cohort effects, and thus, better generalization behavior, reduced feature spaces, or simplified computational implementation and evaluation.

In various embodiments, at least 5 performance metrics, at least 10 performance metrics, at least 15 performance metrics, or at least 20 performance metrics are evaluated to determine whether the challenger model may usurp the champion model in the Production Module.

In various embodiments, between at least 5 and 20 performance metrics, between at least 10 and 15 performance metrics, between at least 5 and 15 performance metrics, or between at least 10 and 20 performance metrics, are evaluated to determine whether the challenger model may usurp the champion model in the Production Module.

In various embodiments, a successful challenger model is deployed automatically to the Production Module.

In various embodiments, a successful challenger model is deployed to the Production Module only after manual review and approval.

In various embodiments, outputs of this module comprise a deployed model.

### C. Production Module

A Production Module may comprise a Product Inference Module and may act as the primary production model pipeline in a production environment to produce raw molecular and clinical data for processing using the feedback loop.

One element of the Production Module may be a Production Inference Module, which may be the model pipeline in a production environment and may produce raw data for ingestion into the feedback loop.

One element of the Production Module may be a Research Ingestion Module which may process clinical metadata and label with quality control metrics, match the clinical metadata with patient molecular data, and push this matched clinical and molecular data to the Research Platform Module.

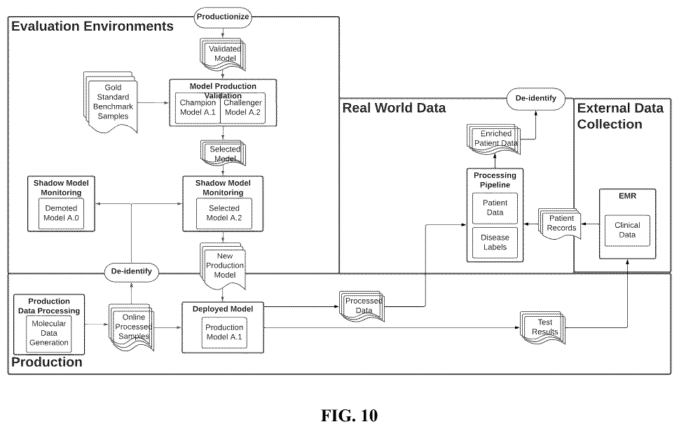

FIG. 10 provides a schema showing the interaction of the Evaluation Module, Production Module, RWD and External Data Ingestion Modules useful for the feedback loops described herein.

In various embodiments, the Production Model comprises a Production Inference Module and a Research Ingestion Module.

In various embodiments, inputs to be processed using the Production Model are selected from a deployed model and processed biological samples.

In certain embodiments, the biological sample is selected from a sample of cell-free nucleic acid, plasma, serum, whole blood, buffy coat, single cell, or tissue.

In various embodiments, patient data comprises biological sample molecular data, clinical data, controlled experimental data and real-world clinical data, or a combination thereof.

In certain embodiments, molecular data comprises information derived from molecules in a biological sample from a subject such as, but not limited to, nucleic acid sequence, length, end point, mid point, methylation status or mutation information; protein sequence, abundance, profile, binding affinity information; autoantibody abundance, profile, diversity information; and metabolite abundance, profile, diversity.

In certain embodiments, molecular data is obtained from a biological sample wherein a predetermined targeted set of biomarkers is targeted for evaluation in the biological sample to provide the molecular data from the biological sample.

In certain embodiments, the predetermined targeted set of biomarkers comprises biomarkers or features of at least 2, at least 3, at least 4, at least 5, at least 6, at least 7, at least 8, at least 9, at least 10, at least 15, or at least 20 cell proliferative disorders.

In some embodiments, the cell proliferative disorder is selected from colorectal, prostate, lung, breast, pancreatic, ovarian, uterine, liver, esophagus, stomach, or thyroid cell proliferation.

In some embodiments, the cell proliferative disorder is selected from colon adenocarcinoma, liver hepatocellular carcinoma, lung adenocarcinoma, lung squamous cell carcinoma, ovarian serious cystadenocarcinoma, pancreatic adenocarcinoma, prostate adenocarcinoma, or rectum adenocarcinoma.

In various embodiments, outputs of this module comprise: processed molecular data, patient test results, or de-identified molecular data.

Notably, molecular data is obtained without associated symptom, disease, progression, or responsiveness labels obtained from clinical or EMR report data and is matched in the external feedback/data collection module.

### D. External Feedback/Data Collection Module

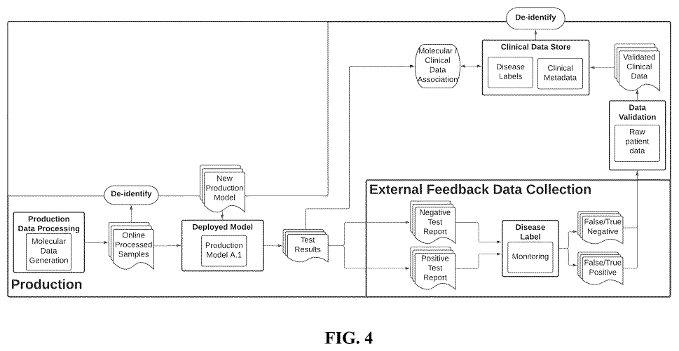

An External Feedback/Data Collection Module may be used to include additional clinical data, labels from medical records such as EHR information to be introduced into the data feedback loop. In certain embodiments, supervised learning techniques construct predictive models by learning from a large number of training examples in which each training example has a label indicating the ground truth output. FIG. 4 provides a schematic showing the operational connection of Production Inference Module and External Feedback and Data Collection Module isolated from the other modules of the feedback loop that may be useful for the feedback loops described herein.

In certain embodiments, Ground Truth data is provided from the External Feedback/Data Collection Module. As used herein for machine learning applications, the term “ground truth” may refer to the accuracy of the training set's classification for supervised learning techniques. Ground truth may be used in statistical models to prove or disprove research hypotheses. The term “ground truthing” may refer to the process of gathering the proper objective (provable) data.

In certain embodiments, the External Feedback/Data Collection Module receives labels from medical records (in EMR records, for example) associated with individuals from whom molecular data is obtained from prior evaluation of biological samples. In certain embodiments, the medical record labels with any newly diagnosed diseases, symptoms, or specifically cancers from a patient are matched to the molecular data associated with that patient. The present Feedback Loops described herein may permit integration of molecular data and later-obtained medical record labels to be processed using the Research Platform Module for creating new classification models or training new classification models as described herein. The collection of a predetermined targeted collection of biomarkers as described herein permits the association of medical record labels with other disease, symptom, or cancer-associated molecular markers.

In certain embodiments, real-world clinical data comprises information derived from a plurality of demographic, physiological, and clinical features, wherein the plurality of demographic, physiological, and clinical features comprises at least two features obtained from demographic, symptomatic, lifestyle, diagnosis, or biochemical variables.

In one embodiment, the demographic variables are selected from age, gender, weight, height, BMI, race, country, geographically determined data such as local air quality, limiting long-term illness, or Townsend deprivation index, and the like. The variable may then be featurized for use in the computerized method or for use as an input to train a computational classification model.

In one embodiment, the symptomatic variables are selected from heartburn, sweating, hemorrhoids, chronic sinusitis, atrial fibrillation, rectal bleeding, abdominal pain, bowel movement change, reflux, constipation, indigestion, hypertension, hematuria, diarrhea, bloating belching, abnormal weight loss, and obesity. The variable may then be featurized for use in the computerized method or for use as an input to train a computational classification model.

In one embodiment, the lifestyle variables are selected from smoking and alcohol use, red meat consumption, and medications such as progestogen, estrogen and other hormonal modulators, hormonal contraceptives, non-steroidal anti-inflammatory and antirheumatic drugs (NSAID), antithrombotic agents, blood glucose lowering drugs excluding insulin, insulin and analogues, immunosuppressants, sulfonamides and trimethoprim antibacterials, or drugs for peptic ulcer and gastroesophageal reflux disease (GERD). The variable may then be featurized for use in the computerized method or for use as an input to train a computational classification model.

In one embodiment, the diagnosis variables are selected from diverticular disease, multiple sclerosis, diabetes 1, diabetes 2, hyperthyroidism, psoriasis, anemia, peripheral arterial disease, AIDS, mental health disorder, inflammatory bowel disease, myocardial infarction, lupus, dementia, heart failure, hepatitis, alcoholic fatty liver disease, cirrhosis, rheumatoid arthritis, acromegaly, hemiplegia, COPD, irritable bowel syndrome, learning disabilities, depression, stroke, and chronic kidney disease. The variable may then be featurized for use in the computerized method or for use as an input to train a computational classification model.

In one embodiment, the biochemical variables are selected from alanine aminotransferase (ALT), albumin, alkaline phosphatase (ALP), aspartate aminotransferase (AST), calcium, carbon dioxide, chloride, cholesterol, creatinine, ferritin, globulin, glucose, high density lipoprotein (HDL), low density lipoprotein (LDL), phosphate, potassium, sodium, triglycerides, urea, and/or uric acid. The variable may then be featurized for use in the computerized method or for use as an input to train a computational classification model.

### E. Research Ingestion Module

In the Research Ingestion Module, clinical metadata and labels may be matched with patient molecular data, EMR data, or other individual data and pushed to the research platform. In certain embodiments, the clinical metadata is subjected to a quality control operation to meet pre-specified quality metrics before matching with patient molecular data.

In various embodiments, inputs to this module are selected from: processed molecular data, disease label, clinical data, and a combination thereof.

In various embodiments, outputs of this module comprise: de-identified, matched patient data, or a combination thereof.

In one embodiment, test results from the Production Inference Module are processed using an External Feedback Data Collection Module and a RWD Module and then processed using the Research Ingestion Module or the Research Platform Module.

The RWD Module provides an optional ingest pathways from the Product Pipeline to provide a secure location for PHI data outside of the Production Environment Module. This data may then be curated, integrated with other RWD production streams, and de-identified to be safely input and used in the Research Platform Module to preserve data security and patient privacy. In some embodiments, Patient Data is processed using the Research Platform Module in a form that is curated, reliable, useful for research efforts, and meets regulatory standards (HIPAA, system integration timelines, etc.) as part of the feedback loop and RWD ingestion and use. In the RWD Module, processed data from the Production Module and Patient Record Information from the External Data Collection Module are received by the RWD Processing Pipeline element. The RWD Processing Pipeline outputs Enriched Patient Data which is then de-identified as described herein before input to a Research Platform Module described herein.

In one embodiment, information within the Clinical Data Store (FIG. 4) or Enriched Patient Data (FIG. 12) is pushed with a predetermined periodicity into the Research Ingestion Module of the Research Platform Module. In certain embodiments, information is de-identified before pushing into the Research Ingestion Module.

In one embodiment, predetermined periodicity is selected from about 1 month, about 3 months, about 6 months, about 9 months, about 12 months, about 18 months, or about 24 months.

In one embodiment, predetermined periodicity occurs by the number of patient data profiles received by the Data Ingestion Module. In some embodiments, the number of patient data profiles is selected from about 100 patient data profiles received, 200 patient data profiles received, 300 patient data profiles received, 400 patient data profiles received, 500 patient data profiles received, 600 patient data profiles received, 700 patient data profiles received, 800 patient data profiles received, 900 patient data profiles received, 1000 patient data profiles received, 1500 patient data profiles received, 2000 patient data profiles received, 2500 patient data profiles received, 3000 patient data profiles received, 3500 patient data profiles received, or 4000 patient data profiles received. A patient data profile may be a collection of clinical or molecular data specific to one patient at one point in time.

### II. Classifiers, Machine Learning Models & Systems

In various examples, molecular and clinical data from biological samples is “featurized” into numerical features corresponding to specified properties of each of the plurality of classes of sample molecules in a biological sample, or labels obtained from clinical data and electronic health records. The features may be used as input datasets to be processed using trained algorithms (e.g., machine learning models or classifiers) to find correlations between molecular and clinical data between patient groups. Examples of such patient groups include presence of diseases or conditions, stages, subtypes, responders vs. non-responders, and progressors vs. non-progressors. In various examples, feature matrices are generated to compare samples obtained from individuals with defined conditions or characteristics. In some embodiments, samples are obtained from healthy individuals, or individuals who do not have any of the defined indications and samples from patients having or exhibiting symptoms of cancer.

In some cases, the samples are associated with the presence of a biological trait, which can be used to train the machine learning model.

In some cases, the biological trait is selected from malignancy, cancer type, cancer stage, cancer classification, metabolic profile, mutation, clinical outcome, drug response, and a combination thereof.

In some embodiments, the biological trait comprises malignancy.

In some embodiments, the biological trait comprises a cancer type.

In some embodiments, the biological trait comprises a cancer stage.

In some embodiments, the biological trait comprises a cancer classification.

In some embodiments, the cancer classification comprises a cancer grade.

In some embodiments, the cancer classification comprises a histological classification.

In some embodiments, the biological trait comprises a metabolic profile.

In some embodiments, the biological trait comprises a mutation.

In some embodiments, the mutation comprises a disease-associated mutation.

In some embodiments, the biological trait comprises a clinical outcome.

In some embodiments, the biological trait comprises a drug response.

As used herein, relating to machine learning and pattern recognition, the term “feature” generally refers to an individual measurable property or characteristic of a phenomenon being observed. The concept of “feature” may be related to that of explanatory variable used in statistical techniques such as, for example, but not limited to, linear regression and logistic regression. Features may be numeric, but structural features such as strings and graphs may be used.

The term “input features” (or “features”), as used herein, generally refers to variables that are used by the trained algorithm (e.g., model or classifier) to predict an output classification (label) of a sample, e.g., a condition, sequence content (e.g., mutations), suggested data collection operations, or suggested treatments. Values of the variables may be determined for a sample and used to determine a classification.

For a plurality of assays, the system may identify feature sets to input into a trained algorithm (e.g., machine learning model or classifier). The system may perform an assay on each molecule class and form a feature vector from the measured values. The system may process the feature vector using the machine learning model and obtain an output classification of whether the biological sample has a specified property.

In various embodiments, immune-derived biological signals in genomic or cell-fee DNA (cfDNA) can be represented as numerical values characteristic of cellular composition (immune cell type of origin for sequence fragments), genes and biological pathways the signals involve, or transcription factor activity (such as transcription factor binding, silencing, or activation).

In some embodiments, the machine learning model outputs a classifier capable of distinguishing between two or more groups or classes of individuals or features in a population of individuals or features of the population. In some embodiments, the classifier model comprises a trained machine learning classifier.

In some embodiments, the informative loci or features of biomarkers in a cancer tissue are assayed to form a profile. Receiver-operating characteristic (ROC) curves may be generated by plotting the performance of a particular feature (e.g., any of the biomarkers described herein and/or any item of additional biomedical information) in distinguishing between two populations (e.g., individuals responding and not responding to a therapeutic agent). In some embodiments, the feature data across the entire population (e.g., the cases and controls) are sorted in ascending order based on the value of a single feature.

In various embodiments, the specified property is selected from healthy vs. cancer, disease subtype, disease stage, progressor vs. non-progressor, and responder vs. non-responder.

### A. Data Analysis

In some examples, the present disclosure provides a system, method, or kit having data analysis realized in software application, computing hardware, or both. In various examples, the analysis application or system comprises at least a data receiving module, a data pre-processing module, a data analysis module (which can operate on one or more types of genomic data), a data interpretation module, or a data visualization module. In some embodiments, the data receiving module can comprise computer systems that connect laboratory hardware or instrumentation with computer systems that process laboratory data. In some embodiments, the data pre-processing module can comprise hardware systems or computer software that performs operations on the data in preparation for analysis. Examples of operations that may be applied to the data in the pre-processing module include affine transformations, denoising operations, data cleaning, reformatting, or subsampling. A data analysis module, which may be specialized for analyzing genomic data from one or more genomic materials, can, for example, take assembled genomic sequences and perform probabilistic and statistical analysis to identify abnormal patterns related to a disease, pathology, state, risk, condition, or phenotype. A data interpretation module can use analysis methods, for example, drawn from statistics, mathematics, or biology, to support understanding of the relation between the identified abnormal patterns and health conditions, functional states, prognoses, or risks. A data visualization module can use methods of mathematical modeling, computer graphics, or rendering to create visual representations of data that can facilitate the understanding or interpretation of results.

In various examples, machine learning methods are applied to distinguish samples in a population of samples. In some embodiments, machine learning methods are applied to distinguish samples between healthy and advanced disease (e.g., adenoma) samples, or between disease stages (e.g., pre-cancerous and cancerous, or between Stage I, Stage II, Stage III, or Stage IV).

In some embodiments, the one or more machine learning operations used to train the prediction engine comprise one or more of: a generalized linear model, a generalized additive model, a non-parametric regression operation, a random forest classifier, a spatial regression operation, a Bayesian regression model, a time series analysis, a Bayesian network, a Gaussian network, a decision tree learning operation, an artificial neural network, a recurrent neural network, a convolutional neural network, a reinforcement learning operation, linear or non-linear regression operations, a support vector machine, a clustering operation, and a genetic algorithm operation.

In various examples, computer processing methods are selected from logistic regression, multiple linear regression (MLR), dimension reduction, partial least squares (PLS) regression, principal component regression, autoencoders, variational autoencoders, singular value decomposition, generative adversarial networks, Fourier bases, wavelets, discriminant analysis, support vector machine, decision tree, classification and regression trees (CART), tree-based methods, random forest, gradient boost tree, logistic regression, matrix factorization, multidimensional scaling (MDS), dimensionality reduction methods, t-distributed stochastic neighbor embedding (t-SNE), multilayer perceptron (MLP), network clustering, neuro-fuzzy, and artificial neural networks.

In some examples, the methods disclosed herein can comprise computational analysis on nucleic acid sequencing data of samples from an individual or from a plurality of individuals.

### B. Classifier Generation

In an aspect, the disclosed systems and methods provide a classifier generated based on feature information derived from methylation sequence analysis from biological samples of cfDNA. The classifier forms part of a predictive engine for distinguishing groups in a population based on sequence features identified in biological samples such as cfDNA.

In some embodiments, a classifier is created by normalizing the sequence information by formatting similar portions of the sequence information into a unified format and a unified scale; storing the normalized sequence information in a columnar database; training a prediction engine by applying one or more one machine learning operations to the stored normalized sequence information, the prediction engine mapping, for a particular population, a combination of one or more features; applying the prediction engine to the accessed field information to identify an individual associated with a group; and classifying the individual into a group.

In some embodiments, classifier metrics are used to assess the strength of classification. Non-limiting examples of classifier metrics are selected from Accuracy, Precision, and Recall, F1 Score, Log Loss/Binary Crossentropy, Categorical Crossentropy, or AUC.

Specificity, as used herein, generally refers to “the probability of a negative test among those who are free from the disease”. Specificity may be calculated by the number of disease-free persons who tested negative divided by the total number of disease-free individuals.

In various examples, the model, classifier, or predictive test has a specificity of at least 40%, at least 45%, at least 50%, at least 55%, at least 60%, at least 65%, at least 70%, at least 75%, at least 80%, at least 85%, at least 90%, at least 95%, or at least 99%.

Sensitivity, as used herein, generally refers to “the probability of a positive test among those who have the disease”. Sensitivity may be calculated by the number of diseased individuals who tested positive divided by the total number of diseased individuals.

In various examples, the model, classifier, or predictive test has a sensitivity of at least 40%, at least 45%, at least 50%, at least 55%, at least 60%, at least 65%, at least 70%, at least 75%, at least 80%, at least 85%, at least 90%, at least 95%, or at least 99%.

### C. Digital Processing Device

In some examples, the subject matter described herein can comprise a digital processing device or use of the same. In some examples, the digital processing device can comprise one or more hardware central processing units (CPU), graphics processing units (GPU), or tensor processing units (TPU) that carry out the device's functions. In some examples, the digital processing device can comprise an operating system configured to perform executable instructions.

In some examples, the digital processing device can optionally be connected a computer network. In some examples, the digital processing device may be optionally connected to the Internet. In some examples, the digital processing device may be optionally connected to a cloud computing infrastructure. In some examples, the digital processing device may be optionally connected to an intranet. In some examples, the digital processing device may be optionally connected to a data storage device.

Non-limiting examples of suitable digital processing devices include server computers, desktop computers, laptop computers, notebook computers, sub-notebook computers, netbook computers, netpad computers, set-top computers, handheld computers, Internet appliances, mobile smartphones, and tablet computers. Suitable tablet computers can comprise, for example, those with booklet, slate, and convertible configurations.

In some examples, the digital processing device can comprise an operating system configured to perform executable instructions. For example, the operating system can comprise software, which may comprise programs and data, which manages the device's hardware and provides services for execution of applications. Non-limiting examples of operating systems include Ubuntu, FreeBSD, OpenBSD, NetBSD®, Linux, Apple® Mac OS X Server®, Oracle® Solaris®, Windows Server®, and Novell® NetWare®. Non-limiting examples of suitable personal computer operating systems include Microsoft® Windows®, Apple® Mac OS X®, UNIX®, and UNIX-like operating systems such as GNU/Linux®. In some examples, the operating system may be provided by cloud computing, and cloud computing resources may be provided by one or more service providers.

In some examples, the device can comprise a storage and/or memory device. The storage and/or memory device may be one or more physical apparatuses used to store data or programs on a temporary or permanent basis. In some examples, the device may be volatile memory and require power to maintain stored information. In some examples, the device may be non-volatile memory and retain stored information when the digital processing device is not powered. In some examples, the non-volatile memory can comprise flash memory. In some examples, the non-volatile memory can comprise dynamic random-access memory (DRAM). In some examples, the non-volatile memory can comprise ferroelectric random-access memory (FRAM). In some examples, the non-volatile memory can comprise phase-change random access memory (PRAM).

In some examples, the device may be a storage device such as, for example, CD-ROMs, DVDs, flash memory devices, magnetic disk drives, magnetic tapes drives, optical disk drives, or cloud computing-based storage. In some examples, the storage and/or memory device may be a combination of devices such as those disclosed herein. In some examples, the digital processing device can comprise a display to send visual information to a user. In some examples, the display may be a cathode ray tube (CRT). In some examples, the display may be a liquid crystal display (LCD). In some examples, the display may be a thin film transistor liquid crystal display (TFT-LCD). In some examples, the display may be an organic light emitting diode (OLED) display. In some examples, on OLED display may be a passive-matrix OLED (PMOLED) or active-matrix OLED (AMOLED) display. In some examples, the display may be a plasma display. In some examples, the display may be a video projector. In some examples, the display may be a combination of devices such as those disclosed herein.

In some examples, the digital processing device can comprise an input device to receive information from a user. In some examples, the input device may be a keyboard. In some examples, the input device may be a pointing device such as, for example, a mouse, trackball, track pad, joystick, game controller, or stylus. In some examples, the input device may be a touch screen or a multi-touch screen. In some examples, the input device may be a microphone to capture voice or other sound input. In some examples, the input device may be a video camera to capture motion or visual input. In some examples, the input device may be a combination of devices such as those disclosed herein.

### D. Non-Transitory Computer-Readable Storage Medium

In some examples, the subject matter disclosed herein can comprise one or more non-transitory computer-readable storage media encoded with a program comprising instructions executable by the operating system of an optionally networked digital processing device. In some examples, a computer-readable storage medium may be a tangible component of a digital processing device. In some examples, a computer-readable storage medium may be optionally removable from a digital processing device. In some examples, a computer-readable storage medium can comprise, for example, CD-ROMs, DVDs, flash memory devices, solid state memory, magnetic disk drives, magnetic tape drives, optical disk drives, cloud computing systems and services, and the like. In some examples, the program and instructions may be permanently, substantially permanently, semi-permanently, or non-transitorily encoded on the media.

### E. Computer Systems

The present disclosure provides computer systems that are programmed to implement methods described herein. FIG. 6 shows a computer system 601 that is programmed or otherwise configured to store, process, identify, or interpret patient data, biological data, biological sequences, and reference sequences. The computer system 601 can process various aspects of patient data, biological data, biological sequences, or reference sequences of the present disclosure. The computer system 601 may be an electronic device of a user or a computer system that is remotely located with respect to the electronic device. The electronic device may be a mobile electronic device.

The computer system 601 comprises a central processing unit (CPU, also “processor” and “computer processor” herein) 605, which may be a single core or multi core processor, or a plurality of processors for parallel processing. The computer system 601 also comprises memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620, and peripheral devices 625 are in communication with the CPU 605 through a communication bus (solid lines), such as a motherboard. The storage unit 615 may be a data storage unit (or data repository) for storing data. The computer system 601 may be operatively coupled to a computer network (“network”) 630 with the aid of the communication interface 620. The network 630 may be the Internet, an internet and/or extranet, or an intranet and/or extranet that is in communication with the Internet. The network 630 in some examples is a telecommunication and/or data network. The network 630 can include one or more computer servers, which can enable distributed computing, such as cloud computing. The network 630, in some examples with the aid of the computer system 601, can implement a peer-to-peer network, which may enable devices coupled to the computer system 601 to behave as a client or a server.

The CPU 605 can execute a sequence of machine-readable instructions, which may be embodied in a program or software. The instructions may be stored in a memory location, such as the memory 610. The instructions may be directed to the CPU 605, which can subsequently program or otherwise configure the CPU 605 to implement methods of the present disclosure. Examples of operations performed by the CPU 605 can include fetch, decode, execute, and writeback.

The CPU 605 may be part of a circuit, such as an integrated circuit. One or more other components of the system 601 may be included in the circuit. In some examples, the circuit is an application specific integrated circuit (ASIC).

The storage unit 615 can store files, such as drivers, libraries, and saved programs. The storage unit 615 can store user data, e.g., user preferences and user programs. The computer system 601 in some examples can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 601 via the network 630.

Methods as described herein may be implemented by way of machine (e.g., computer processor) executable code stored on an electronic storage location of the computer system 601, such as, for example, on the memory 610 or electronic storage unit 615. The machine-executable or machine-readable code may be provided in the form of software. During use, the code may be executed by the processor 605. In some examples, the code may be retrieved from the storage unit 615 and stored on the memory 610 for ready access by the processor 605. In some examples, the electronic storage unit 615 may be precluded, and machine-executable instructions are stored on memory 610.

The code may be pre-compiled and configured for use with a machine having a processer adapted to execute the code or may be interpreted or compiled during runtime. The code may be supplied in a programming language that may be selected to enable the code to execute in a pre-compiled, interpreted, or as-compiled fashion.

Aspects of the systems and methods provided herein, such as the computer system 601, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements comprises optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, a nucleic acid sequence, an enriched nucleic acid sample, a methylation profile, an expression profile, and an analysis of a methylation or expression profile. Examples of UI's include, without limitation, a graphical user interface (GUI) and web-based user interface.

Methods and systems of the present disclosure may be implemented by way of one or more algorithms. An algorithm may be implemented by way of software upon execution by the central processing unit 605. The algorithm can, for example, store, process, identify, or interpret patient data, biological data, biological sequences, and reference sequences.

While certain examples of methods and systems have been shown and described herein, one of skill in the art will realize that these are provided by way of example only and not intended to be limiting within the specification. Numerous variations, changes, and substitutions will now occur to those skilled in the art without departing from the scope described herein. Furthermore, it shall be understood that all aspects of the described methods and systems are not limited to the specific depictions, configurations or relative proportions set forth herein which depend upon a variety of conditions and variables and the description is intended to include such alternatives, modifications, variations, or equivalents.

In some examples, the subject matter disclosed herein can include at least one computer program or use of the same. A computer program can a sequence of instructions, executable in the digital processing device's CPU, GPU, or TPU, written to perform a specified task. Computer-readable instructions may be implemented as program modules, such as functions, objects,

Application Programming Interfaces (APIs), data structures, and the like, that perform particular tasks or implement particular abstract data types. In light of the disclosure provided herein, a computer program may be written in various versions of various languages.

The functionality of the computer-readable instructions may be combined or distributed in various environments. In some examples, a computer program can include one sequence of instructions. In some examples, a computer program can include a plurality of sequences of instructions. In some examples, a computer program may be provided from one location. In some examples, a computer program may be provided from a plurality of locations. In some examples, a computer program can include one or more software modules. In some examples, a computer program can include, in part or in whole, one or more web applications, one or more mobile applications, one or more standalone applications, one or more web browser plug-ins, extensions, add-ins, or add-ons, or a combination thereof.

In some examples, the computer processing may be a method of statistics, mathematics, biology, or any combination thereof. In some examples, the computer processing method comprises a dimension reduction method such as, for example, logistic regression, dimension reduction, principal component analysis, autoencoders, singular value decomposition, Fourier bases, singular value decomposition, wavelets, discriminant analysis, support vector machine, tree-based methods, random forest, gradient boost tree, logistic regression, matrix factorization, network clustering, or neural network such as convolutional neural networks.

In some embodiments, the computer processing method comprises a supervised machine learning method such as, for example, a regression, support vector machine, tree-based method, or network. In supervised learning approaches, a group of samples from two or more groups can be analyzed or processed with a statistical classification method. Sequence or expression level can be used as a basis for classifier that differentiates between the two or more groups. A new sample can then be analyzed or processed so that the classifier can associate the new sample with one of the two or more groups. Classification using supervised methods can be performed by the following methodology:
1. Gather a training set. A training set can comprise, for example, sequence information from nucleic acid molecules sequenced herein.
2. Determine the input “feature” representation of the learned function. The accuracy of the learned function may depend on how the input object is represented. The input object may be transformed into a feature vector, which contains a number of features that are descriptive of the object.
3. Determine the structure of the learned function and corresponding learning algorithm. A learning algorithm may be chosen, e.g., artificial neural networks, decision trees, Bayes classifiers, or support vector machines. The learning algorithm may be used to build the classifier.
4. Build the classifier (e.g., classification model). The learning algorithm may be run on the gathered training set. Parameters of the learning algorithm may be adjusted by optimizing performance on a subset (called a validation set) of the training set, or via cross-validation. After parameter adjustment and learning, the performance of the algorithm may be measured on a test set of naive samples that is separate from the training set. The built model can involve feature coefficients or importance measures assigned to individual features.

Once the classifier (e.g., classification model) is determined as described above (e.g., “trained”), the classifier can be used to classify a sample.

In some embodiments, the computer processing method comprises an unsupervised machine learning method such as, for example, clustering, network, principal component analysis, or matrix factorization.

### F. Databases

In some examples, the subject matter disclosed herein can comprise one or more databases, or use of the same to store patient data, clinical data, metadata, molecular data, biological data, biological sequences, or reference sequences. Reference sequences may be derived from a database. In view of the disclosure provided herein, many databases may be suitable for storage and retrieval of the sequence information. In some examples, suitable databases can comprise, for example, relational databases, non-relational databases, object-oriented databases, object databases, entity-relationship model databases, associative databases, and XML databases. In some examples, a database may be internet-based. In some examples, a database may be web-based. In some examples, a database may be cloud computing-based. In some examples, a database may be based on one or more local computer storage devices.

In an aspect, the present disclosure provides a non-transitory computer-readable medium comprising instructions that direct a processor to carry out a method disclosed herein.

In an aspect, the present disclosure provides a computing device comprising the computer-readable medium.

In another aspect, the present disclosure provides a system for performing classifications of biological samples comprising:
a) a receiver to receive a plurality of training samples, each of the plurality of training samples having a plurality of classes of molecules, wherein each of the plurality of training samples comprises one or more defined labels;
b) a feature module to identify a set of features corresponding to an assay that are operable to be processed using the machine learning model for each of the plurality of training samples, wherein the set of features correspond to properties of molecules in the plurality of training samples, wherein for each of the plurality of training samples, the system is operable to subject a plurality of classes of molecules in the training sample to a plurality of different assays to obtain sets of measured values, wherein each set of measured values is from one assay applied to a class of molecules in the training sample, wherein a plurality of sets of measured values are obtained for the plurality of training samples;
c) an analysis module to analyze the sets of measured values to obtain a training vector for the training sample, wherein the training vector comprises feature values of the N set of features of the corresponding assay, each feature value corresponding to a feature and comprising one or more measured values, wherein the training vector is formed using at least one feature from at least two of the N sets of features corresponding to a first subset of the plurality of different assays;
d) a labeling module to inform the system on the training vectors using parameters of the machine learning model to obtain output labels for the plurality of training samples;
e) a comparator module to compare the output labels to the defined labels of the training samples;
f) a training module to iteratively search for optimal values of the parameters as part of training the machine learning model based on the comparing the output labels to the defined labels of the training samples; and
g) an output module to provide the parameters of the machine learning model and the set of features for the machine learning model.

### III. Systems

The diagnostic feedback loops described herein have utility in systems of medical screening, diagnosis, prognosis, treatment determination, and disease monitoring. In various embodiments, the feedback loops described herein are modified according to necessary parameters to suit the requisite needs of a system employing the described feedback loops and to accomplish a predetermined activity.

In another aspect, provided herein is a system comprising a data feedback loop, wherein the data feedback loop comprises:
a research platform module that trains or re-trains a diagnostic classifier;
a production module that produces input data, wherein the production module comprises the diagnostic classifier; and
an external feedback/data collection module that receives data from real-world execution of the diagnostic classifier, wherein the external feedback/data collection module is operatively linked to research platform module; and
a computing device comprising at least one computer processor, an operating system configured to perform executable instructions, a memory, and a computer program comprising instructions executable by the computing device to provide a computer application for executing the data feedback loop.

In one embodiment, the data feedback loop comprises an evaluation/deployment module that provides productionizing of a validated model to prepare for deployment, wherein the evaluation/deployment module is operatively linked between the research platform module and the production module.

In one embodiment, the data feedback loop comprises a data ingestion module that ingests data, wherein the data ingestion module is operatively linked between the external feedback/data collection module and the research platform module.

In one embodiment, the classifier model comprises a machine learning classifier.

In another aspect, provided herein is a non-transitory computer-readable medium comprising machine-executable code that, upon execution by one or more computer processors, implements a method for improving a diagnostic classifier model, the method comprising:
a) obtaining molecular or clinical data from an individual sample associated with a presence or absence of a characteristic of a disease or disorder requiring classification;
b) inputting the molecular or clinical data into a data feedback loop system comprising:
   i) a research platform module that trains or re-trains a diagnostic classifier;
   ii) a production module that produces input data, wherein the production module comprises the diagnostic classifier; and
   iii) an external feedback/data collection module that receives data from real-world execution of the diagnostic classifier; and
c) re-training the diagnostic classifier to improve one or more classification metrics of the diagnostic classifier.

In one embodiment, the data feedback loop comprises an evaluation/deployment module that provides productionizing of a validated model to prepare for deployment, wherein the evaluation/deployment module is operatively linked between the research platform module and the production module.

In one embodiment, the data feedback loop comprises a data ingestion module that ingests data, wherein the data ingestion module is operatively linked between the external feedback/data collection module and the research platform module.

In one embodiment, the classifier model comprises a machine learning classifier.

In another aspect, provided herein is non-transitory computer-readable medium comprising machine-executable code that, upon execution by one or more computer processors, implements a method for creating a diagnostic classifier, the method comprising:
a) obtaining molecular or clinical data from an individual sample associated with a presence or absence of a characteristic of a disease or disorder requiring classification;
b) inputting the molecular or clinical data into a data feedback loop system comprising:
   i) a research platform module that trains or re-trains a diagnostic classifier;
   ii) a production module that produces input data, wherein the production module comprises the diagnostic classifier; and
   iii) an external feedback/data collection module that receives data from real-world execution of the diagnostic classifier; and
c) training a diagnostic classifier to provide one or more classification metrics capable of classifying a population of individuals for the disease or disorder.

In one embodiment, the classifier model comprises a machine learning classifier.

In one embodiment, the data feedback loop comprises an evaluation/deployment module that productionizes a validated model to prepare for deployment, wherein the evaluation/deployment module is operatively linked between the research platform module and the production module.

In one embodiment, the data feedback loop comprises a data ingestion module that ingests data, wherein the data ingestion module is operatively linked between the external feedback/data collection module and the research platform module.

In another aspect, provided herein is a system comprising a computing device comprising a computer processor, an operating system configured to perform executable instructions, a memory, and a computer program comprising instructions executable by the computer processor to provide a computer application for creating a diagnostic classifier comprising:
a) obtaining molecular or clinical data from an individual sample associated with a presence or absence of a characteristic of a disease or disorder requiring classification;
b) inputting the molecular or clinical data into a data feedback loop system comprising:
   i) a research platform module that trains or re-trains a diagnostic classifier;
   ii) a production module that produces input data, the production module; and
   iii) an external feedback/data collection module that receives data from real-world execution of the diagnostic classifier; and
c) training a diagnostic classifier to provide one or more classification metrics capable of classifying a population of individuals for the disease or disorder.

In one embodiment, the data feedback loop comprises an evaluation/deployment module that productionizes a validated model to prepare for deployment, wherein the evaluation/deployment module is operatively linked between the research platform module and the production module.

In one embodiment, the classifier model comprises a machine learning classifier.

In one embodiment, the data feedback loop comprises a data ingestion module that ingests data, wherein the data ingestion module is operatively linked between the external feedback/data collection module and the research platform module.

In another aspect, provided herein is a system comprising a computing device comprising at least one computer processor, an operating system configured to perform executable instructions, a memory, and a computer program comprising instructions executable by the computing device to provide a computer application for improving a diagnostic classifier model comprising:
a) obtaining molecular and/or clinical data from an individual sample associated with the presence or absence of a specified property of a disease or disorder requiring classification;
b) processing the molecular and/or clinical data from the individual using a data feedback loop, wherein the data feedback loop comprises:
   i) a research platform module that trains or re-trains a diagnostic classifier;
   ii) a production module that produces input data, wherein the production module comprises the diagnostic classifier; and
   iii) an external feedback/data collection module that receives data from real-world execution of the diagnostic classifier; and
c) re-training the diagnostic classifier to improve one or more classification metrics of the diagnostic classifier.

In one embodiment, the data feedback loop comprises an evaluation/deployment module that productionizes a validated model to prepare for deployment, wherein the evaluation/deployment module is operatively linked between the research platform module and the production module.

In one embodiment, the classifier model comprises a machine learning classifier.

In one embodiment, the data feedback loop comprises a data ingestion module that ingests data, wherein the data ingestion module is operatively linked between the external feedback/data collection module and the cohort selection and retraining module.

In some embodiments, provided herein is a classifier in a system for detecting a cell proliferative disorder, the classifier comprising:
a) a computer-readable medium comprising a classifier operable to classify the subjects based on a feedback loop described herein; and
b) one or more processors for executing instructions stored on the computer-readable medium.

In some embodiments, the system comprises a classification circuit that is configured as a machine learning classifier selected from a deep learning classifier, a neural network classifier, a linear discriminant analysis (LDA) classifier, a quadratic discriminant analysis (QDA) classifier, a support vector machine (SVM) classifier, a random forest (RF) classifier, a linear kernel support vector machine classifier, a first or second order polynomial kernel support vector machine classifier, a ridge regression classifier, an elastic net algorithm classifier, a sequential minimal optimization algorithm classifier, a naive Bayes algorithm classifier, and principal component analysis classifier.

In some embodiments, the computer-readable medium comprises a non-transitory computer-readable medium comprising machine-executable code that, upon execution by one or more computer processors, implements any of the methods above or elsewhere herein.

In some embodiments, the system comprises one or more computer processors and computer memory coupled thereto. The computer memory comprises machine-executable code that, upon execution by the one or more computer processors, implements any of the methods described herein.

### IV. Methods of Use

The diagnostic feedback loops described herein may have utility in methods of medical screening, diagnosis, prognosis, treatment determination, and disease monitoring. In various embodiments, the feedback loops described herein are modified according to necessary parameters to suit the requisite needs of a method of use and to accomplish a predetermined activity.

In one embodiment, the data feedback loop described herein is used to refresh, revise, or update an existing classification model that has been deployed in a Production Module. In this embodiment, parameters and weights of a model are modified based on additional input data incorporated into the feedback loop.

In another embodiment, the data feedback loop described herein may be used to refresh, revise, or update an existing classification model that has been deployed in the Production Module. In this embodiment, the architecture of a model is modified based on additional input data incorporated into the feedback loop. In certain embodiments, the composition of analytical tools employed in the model is modified in response to the additional input data.

In various embodiments, incorporation of machine learning tools may be selected from: deep learning models, neural networks (e.g., deep learning neural networks), kernel-based regressions, adaptive basis regression or classification, Bayesian methods, ensemble methods, logistic regression and extensions, Gaussian processes, support vector machines (SVMs), a probabilistic model, and a probabilistic graphical model.

In some embodiments, the system comprises a classification circuit that is configured as a machine learning classifier selected from a deep learning classifier, a neural network classifier, a linear discriminant analysis (LDA) classifier, a quadratic discriminant analysis (QDA) classifier, a support vector machine (SVM) classifier, a random forest (RF) classifier, a linear kernel support vector machine classifier, a first or second order polynomial kernel support vector machine classifier, a ridge regression classifier, an elastic net algorithm classifier, a sequential minimal optimization algorithm classifier, a naive Bayes algorithm classifier, and a principal component analysis classifier.

In other embodiments, the composition of analyte data is modified and molecular data from additional analytes may be incorporated into the model selected from DNA, RNA, polynucleotide, polypeptide, carbohydrate, or metabolite molecular data. In one embodiment, the DNA molecular data is cfDNA molecular data. In another embodiment, the DNA molecular data is methylation status data of the DNA.

In other embodiments, the model is modified to increase classification of additional clinical indications. In one embodiment, a model for classifying one type of cancer is modified to classify two or more types of cancer.

In another aspect, provided herein is a method for improving a diagnostic machine learning classifier, the method comprising:
a) obtaining molecular and/or clinical data from an individual sample associated with the presence or absence of a specified property of a disease or disorder requiring classification;
b) inputting the molecular and/or clinical data from the individual into a data feedback loop comprising:
   i) a research platform module that trains or re-trains a diagnostic classifier;
   ii) a production module that produces input data, wherein the production module comprises the diagnostic classifier; and
   iii) an external feedback/data collection module that receives data from real-world execution of the diagnostic classifier; and
c) re-training the diagnostic classifier to improve one or more classification metrics of the diagnostic classifier.

In one embodiment, the data feedback loop comprises an evaluation/deployment module that provides productionizing of a validated model to prepare for deployment, wherein the evaluation/deployment module is operatively linked between the research platform module and the production module.

In one embodiment, the data feedback loop comprises a data ingestion module that ingests data, wherein the data ingestion module is operatively linked between the external feedback/data collection module and the research platform module.

In one embodiment, the machine learning classifier is trained on a set of training biological samples wherein the set of training biological samples consists of a first subset of the training biological samples identified as having a specified property and a second subset of the training biological samples identified as not having the specified property, wherein the machine learning classifier provides an output classification of whether the biological sample has the specified property, thereby distinguishing a population of individuals having the specified property. In various examples, the specified property can be a clinically-diagnosed disorder.

In various examples, the clinically-diagnosed disorder is cancer. As examples, the cancer is colorectal cancer, liver cancer, lung cancer, pancreatic cancer, or breast cancer.

In some examples, the specified property is clinical staging of a disease or clinically-diagnosed disorder.

In some examples, the specified property is responsiveness to a treatment.

In one example, the specified property comprises a continuous measurement of a patient trait or phenotype at two or more points in time.

In another aspect, provided herein is a method of creating a new diagnostic classifier model, the method comprising:
a) obtaining molecular and/or clinical data from an individual associated with the presence or absence of a characteristic of a disease or disorder requiring classification;
b) inputting the molecular and/or clinical data from the individual into a data feedback loop comprising:
   i) a research platform module that trains or re-trains a diagnostic classifier;
   ii) a production module that produces input data, the production module; and
   iii) an external feedback/data collection module that receives data from real-world execution of the diagnostic classifier; and
c) training diagnostic classifier to provide one or more classification metrics capable of classifying a population of individuals for the disease or disorder.

In one embodiment, the data feedback loop comprises an evaluation/deployment module that provides productionizing of a validated model to prepare for deployment, and the evaluation/deployment module is operatively linked between the research platform module and the production module.

In one embodiment, the data feedback loop comprises a data ingestion module that ingests data, wherein the data ingestion module is operatively linked between the external feedback/data collection module and the research platform module.

In one embodiment, the classifier model comprises a machine learning classifier.

Methods and systems provided herein may perform predictive analytics using artificial intelligence-based approaches to analyze acquired data from a subject (patient) to generate an output of diagnosis of the subject having a cell proliferative disorder such as cancer. For example, the application may apply a prediction algorithm to the acquired data to generate the diagnosis of the subject having the cancer. The prediction algorithm may comprise an artificial intelligence-based predictor, such as a machine learning-based predictor, configured to process the acquired data to generate the diagnosis of the subject having the cancer.

The machine learning predictor may be trained using datasets, e.g., datasets generated by performing methylation assays using the signature panels on biological samples of individuals from one or more sets of cohorts of patients having cancer as inputs and diagnosis (e.g., staging and/or tumor fraction) outcomes of the subjects as outputs to the machine learning predictor.

Training datasets (e.g., datasets generated by performing methylation assays using the signature panels on biological samples of individuals) may be generated from, for example, one or more sets of subjects having common characteristics (features) and outcomes (labels). Training datasets may comprise a set of features and labels corresponding to the features relating to diagnosis. Features may comprise characteristics such as, for example, certain ranges or categories of cfDNA assay measurements, such as counts of cfDNA fragments in a biological sample obtained from a healthy and disease samples that overlap or fall within each of a set of bins (genomic windows) of a reference genome. For example, a set of features collected from a given subject at a given time point may collectively serve as a diagnostic signature, which may be indicative of an identified cancer of the subject at the given time point. Characteristics may also comprise labels indicating the subject's diagnostic outcome, such as for one or more cancers.

Labels may comprise outcomes such as, for example, a predicted or validated diagnosis (e.g., staging and/or tumor fraction) outcomes of the subject. Outcomes may comprise a characteristic associated with the cancers in the subject. For example, characteristics may be indicative of the subject having one or more cancers.

Training sets (e.g., training datasets) may be selected by random sampling of a set of data corresponding to one or more sets of subjects (e.g., retrospective and/or prospective cohorts of patients having or not having one or more cancers). Alternatively, training sets (e.g., training datasets) may be selected by proportionate sampling of a set of data corresponding to one or more sets of subjects (e.g., retrospective and/or prospective cohorts of patients having or not having one or more cancers). Training sets may be balanced across sets of data corresponding to one or more sets of subjects (e.g., patients from different clinical sites or trials). The machine learning predictor may be trained until certain predetermined conditions for accuracy or performance are satisfied, such as exhibiting particular diagnostic accuracy measures. For example, the diagnostic accuracy measure may correspond to prediction of a diagnosis, staging, or tumor fraction of one or more cancers in the subject.

Examples of diagnostic accuracy measures may include sensitivity, specificity, positive predictive value (PPV), negative predictive value (NPV), accuracy, and area under the curve (AUC) of a Receiver Operating Characteristic (ROC) curve corresponding to the diagnostic accuracy of detecting or predicting the cancer.

After using a trained algorithm to process the dataset, the cancer may be identified or monitored in the subject. The identification may be based at least in part on quantitative measures of sequence reads of the dataset at a panel of cancer-associated genomic loci (e.g., quantitative measures of RNA transcripts or DNA at the cancer-associated genomic loci).

Non-limiting examples of cancers that can be inferred by the disclosed methods and systems include acute lymphoblastic leukemia (ALL), acute myeloid leukemia (AML), adrenocortical carcinoma, Kaposi Sarcoma, anal cancer, basal cell carcinoma, bile duct cancer, bladder cancer, bone cancer, osteosarcoma, malignant fibrous histiocytoma, brain stem glioma, brain cancer, craniopharyngioma, ependymoblastoma, ependymoma, medulloblastoma, medulloepithelioma, pineal parenchymal tumor, breast cancer, bronchial tumor, Burkitt lymphoma, Non-Hodgkin lymphoma, carcinoid tumor, cervical cancer, chordoma, chronic lymphocytic leukemia (CLL), chronic myelogenous leukemia (CML), colon cancer, colorectal cancer, cutaneous T-cell lymphoma, ductal carcinoma in situ, endometrial cancer, esophageal cancer, Ewing Sarcoma, eye cancer, intraocular melanoma, retinoblastoma, fibrous histiocytoma, gallbladder cancer, gastric cancer, glioma, hairy cell leukemia, head and neck cancer, heart cancer, hepatocellular (liver) cancer, Hodgkin lymphoma, hypopharyngeal cancer, kidney′ cancer, laryngeal cancer, lip cancer, oral cavity cancer, lung cancer, non-small cell carcinoma, small cell carcinoma, melanoma, mouth cancer, myelodysplastic syndromes, multiple myeloma, medulloblastoma, nasal cavity cancer, paranasal sinus cancer, neuroblastoma, nasopharyngeal cancer, oral cancer, oropharyngeal cancer, osteosarcoma, ovarian cancer, pancreatic cancer, papillomatosis, paraganglioma, parathyroid cancer, penile cancer, pharyngeal cancer, pituitary tumor, plasma cell neoplasm, prostate cancer, rectal cancer, renal cell cancer, rhabdomyosarcoma, salivary gland cancer, Sezary syndrome, skin cancer, small intestine cancer, soft tissue sarcoma, squamous cell carcinoma, testicular cancer, throat cancer, thymoma, thyroid cancer, urethral cancer, uterine cancer, uterine sarcoma, vaginal cancer, vulvar cancer, Waldenstrom macroglobulinemia, and Wilms Tumor, and a combination thereof.

The cancer may be identified in the subject at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The accuracy of identifying the cancer by the trained algorithm may be calculated as the percentage of independent test samples (e.g., subjects having or exhibiting symptoms of cancer or subjects with negative clinical test results for the cancer) that are correctly identified or classified as having or not having the cancer.

The cancer may be identified in the subject with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the cancer using the trained algorithm may be calculated as the percentage of cell-free biological samples identified or classified as having the cancer that correspond to subjects that truly have the cancer.

The cancer may be identified in the subject with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the cancer using the trained algorithm may be calculated as the percentage of cell-free biological samples identified or classified as not having the cancer that correspond to subjects that truly do not have the cancer.

The cancer may be identified in the subject with a clinical sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the cancer using the trained algorithm may be calculated as the percentage of independent test samples associated with presence of the cancer (e.g., subjects having or exhibiting symptoms of the cancer) that are correctly identified or classified as having the cancer.

The cancer may be identified in the subject with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the cancer using the trained algorithm may be calculated as the percentage of independent test samples associated with absence of the cancer (e.g., subjects with negative clinical test results for the colorectal cancer) that are correctly identified or classified as not having the cancer.

In some embodiments, the trained algorithm or classifier model may determine that the subject is at risk of colorectal cancer of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

The trained algorithm or classifier model may determine that the subject is at risk of cancer at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more.

### Treatment Responsiveness

The predictive classifiers, systems, and methods described herein may be applied toward classifying populations of individuals for a number of clinical applications. Examples of such clinical applications include, detecting early-stage cancer, diagnosing cancer, classifying cancer to a particular stage of disease, determining responsiveness or resistance to a therapeutic agent for treating cancer.

The methods and systems described herein may be applied to characteristics of a cell proliferative disorder, such as grade and stage. Therefore, the feedback loops described herein may be used in the present systems and methods to predict responsiveness of cancer therapeutics across different cancer types in different tissues and classifying individuals based on treatment responsiveness. In some embodiments, the classifiers described herein are capable of stratifying a group of individuals into treatment responders and non-responders.

The present disclosure also provides a method for determining the efficacy of a drug designed to treat a disease class, the method comprising: obtaining a sample from an individual having the disease class; subjecting the sample to the drug; assessing the response; and using a computer model built with a weighted voting scheme, classifying the drug-exposed sample into a class of the disease as a function of relative response of the sample with respect to that of the model.

The present disclosure also provides a method for determining the efficacy of a drug designed to treat a disease class, wherein an individual has been subjected to the drug, the method comprising: obtaining a sample from the individual subjected to the drug; assessing the sample for the level of gene expression for at least one gene; and using a model built with a weighted voting scheme, classifying the sample into a class of the disease such as evaluating the sample as compared the model.

In an aspect, the systems and methods described herein that relate to classifying a population based on treatment responsiveness refer to cancers that are treated with chemotherapeutic agents of the classes DNA damaging agents, DNA repair target therapies, inhibitors of DNA damage signaling, inhibitors of DNA damage induced cell cycle arrest and inhibition of processes indirectly leading to DNA damage, but not limited to these classes. Each of these chemotherapeutic agents may be considered a “DNA-damage therapeutic agent” as the term is used herein.

Based on a patient's molecular or clinical data the patient may be classified into high-risk and low-risk patient groups, such as patient with a high or low risk of clinical relapse, and the results may be used to determine a course of treatment. For example, a patient determined to be a high-risk patient may be treated with adjuvant chemotherapy after surgery. For a patient deemed to be a low-risk patient, adjuvant chemotherapy may be withheld after surgery. Accordingly, the present disclosure provides, in certain aspects, a method for preparing a gene expression profile of a colon cancer tumor that is indicative of risk of recurrence.

In various examples, the classifiers described herein are capable of stratifying a population of individuals between responders and non-responders to treatment.

In another aspect, methods disclosed herein may be applied to clinical applications involving the detection or monitoring of cancer.

In some embodiments, methods disclosed herein may be applied to determine or predict response to treatment.

In some embodiments, methods disclosed herein may be applied to monitor or predict tumor load.

In some embodiments, methods disclosed herein may be applied to detect and/or predict residual tumor post-surgery.

In some embodiments, methods disclosed herein may be applied to detect and/or predict minimal residual disease post-treatment.

In some embodiments, methods disclosed herein may be applied to detect or predict relapse.

In an aspect, methods disclosed herein may be applied as a secondary screen.

In an aspect, methods disclosed herein may be applied as a primary screen.

In an aspect, methods disclosed herein may be applied to monitor cancer development.

In an aspect, methods disclosed herein may be applied to monitor or predict cancer.

Upon identifying the subject as having the cancer, the subject may be optionally provided with a therapeutic intervention (e.g., prescribing an appropriate course of treatment to treat the cancer of the subject). The therapeutic intervention may comprise a prescription of an effective dose of a drug, a further testing or evaluation of the cancer, a further monitoring of the cancer, or a combination thereof. If the subject is currently being treated for the cancer with a course of treatment, the therapeutic intervention may comprise a subsequent different course of treatment (e.g., to increase treatment efficacy due to non-efficacy of the current course of treatment).

The therapeutic intervention may comprise recommending the subject for a secondary clinical test to confirm a diagnosis of the cancer. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, a FIT test, an FOBT test, or a combination thereof.

The quantitative measures of sequence reads of the dataset at the panel of T cell receptor or B cell receptor repertoire sequences (e.g., quantitative measures of RNA transcripts or DNA of T cell receptor or B cell receptor repertoire sequences) may be assessed over a duration of time to monitor a patient (e.g., subject who has cancer or who is being treated for cancer). In such cases, the quantitative measures of the dataset of the patient may change during the course of treatment. For example, the quantitative measures of the dataset of a patient with decreasing risk of the cancer due to an effective treatment may shift toward the profile or distribution of a healthy subject (e.g., a subject without cancer). Conversely, for example, the quantitative measures of the dataset of a patient with increasing risk of the cancer due to an ineffective treatment may shift toward the profile or distribution of a subject with higher risk of the cancer or a more advanced cancer.

The cancer of the subject may be monitored by monitoring a course of treatment for treating the cancer of the subject. The monitoring may comprise assessing the cancer of the subject at two or more time points.

The methods can be understood by the following:

A method for assessing a second patient sample comprising: (a) assessing a first patient sample; (b) generating a first patient sample result; (c) obtaining molecular and/or clinical data from the first patient that is characteristic of a disease or disorder; (d) processing the molecular and/or clinical data from the first patient using a data feedback loop; (e) re-training a machine learning classifier to improve one or more classification metrics to create an improved classification metric; (f) assessing a second patient sample, wherein assessing the second patient sample comprises utilizing the improved characteristic in (b) to generate a second patient sample result.

A method for assessing a first and second patient sample comprising: (a) assessing a first patient sample and generating a first result wherein the first result is based on a classification metric; (b) updating the classification metric with molecular data, clinical data, controlled experimental data, real-world clinical data, or a combination thereof; (c) re-training the machine learning classifier to improve the classification matric based on the data in (b) to generate an improved classification metric; and (d) assessing a second patient sample and generating a second result wherein the second result is based on the improved classification metric.

A method for assessing a first and second patient sample comprising: (a) assessing the first patient sample for molecular data, clinical data, controlled experimental data, real-world clinical data, or a combination thereof; (b) utilizing the data from the first patient sample to train a classifier to create an improved classification metric; and (c) utilizing the improved classification metric to assess the second patient sample.

### Examples

### Example 1: Use of a Rwd Feedback Loop to Improve Machine Learning Classification Model of Early Colorectal Cancer Detection

An order may be submitted to the production system via a participating hospital or clinical. The order comprises clinical data (e.g., age, sex, and other PHI). A patient's blood may be collected, and a tube of the blood may be processed through a machine learning production inference module of a system disclosed herein, thereby producing processed molecular data (Bam files and protein data) and a test result. A primary care physician may use this test result to recommend a diagnostic colonoscopy. The resulting pathology report from this diagnostic test may be sent back to the production system via an EHR integration, along with additional clinical information. A RWD ingestion module may then be used to de-identify this clinical data, fetch corresponding molecular data, and push the data into the Research Platform for further processing.

As soon as molecular and clinical data is received, the feedback loop automatically processes and re-formats the data to store within a data warehouse as a dataset. Stored datasets may then be curated and selected based on quality pre-specifications, identified as a “RWD training class.” These selected datasets may be featurized and then processed through a model retraining pipeline along with previous training classes from previous studies. The model retraining pipeline may re-fit parameters and update the existing model with new data.

Finally, this new model may be evaluated on separate hold out validation sets to compare performance with the current deployed model. If the candidate model surpasses the performance of the deployed model, the model may be productionized and then passed to an evaluation environment for validation. Candidate models may be run against a gold standard benchmark dataset for final confirmation. The model may then be pushed to a staging environment where the models can be used to process de-identified sample data to build confidence in the new model. Once this confidence level is established and appropriate regulatory and other quality requirements are satisfied, this new model may replace the deployed model and may be the model that runs inference on patient blood samples.

1.-10. (canceled)

11. A data feedback loop system comprising:
a) a cohort selection and retraining module that selects classes of training samples for a classification model or re-trains the classification model;
b) a product inference module that produces raw data for ingestion into the data feedback loop system; and
c) an external feedback/data collection module that receives data from real-world execution of the classification model.

12. The data feedback loop system of claim 11, wherein the external feedback/data collection module is operatively linked to the cohort selection and retraining module.

13. The data feedback loop system of claim 11, wherein the cohort selection and retraining module further comprises a training module that trains the classification model.

14. The data feedback loop system of claim 11, wherein the classification model is trained using a federated learning approach.

15. The data feedback loop system of claim 11, wherein the classification model is trained using an active learning approach.

16. The data feedback loop system of claim 11, further comprising an evaluation/deployment module that productionizes a validated model to prepare for deployment, wherein the evaluation/deployment module is operatively linked between the cohort selection and retraining module and the product inference module.

17. The data feedback loop system of claim 16, wherein data flows from the evaluation/deployment module to the product inference module and back to the evaluation/deployment module or forward to the external feedback/data collection module.

18.-19. (canceled)

20. The data feedback loop system of claim 11, wherein the cohort selection and retraining module further comprises: 1) an input selected from a) de-identified patient data matched with a sample, b) feedback loop batching specifications, c) ingested data quality specifications, and d) a combination thereof; and 2) an output of a validated classification model.

21. The data feedback loop system of claim 11, further comprising a data ingestion module that ingests data, wherein the data ingestion module is operatively linked between the external feedback/data collection module and the cohort selection and retraining module.

22. The data feedback loop system of claim 11, further comprising a research ingestion module that processes clinical metadata or labels with quality control metrics, matches the clinical metadata with patient molecular data, or pushes the matched clinical metadata and molecular data to the research platform module, wherein the research ingestion module is operatively linked between the external feedback/data collection module and the cohort selection and retraining module.

23. The data feedback loop system of claim 22, wherein the research ingestion module further comprises: 1) an input selected from: a) processed sample molecular data, b) disease and clinical condition labels, c) clinical data, and d) a combination thereof; and 2) an output of de-identified patient data matched with a sample.

24. The data feedback loop system of claim 23, wherein the input comprises de-identified patient data matched with a sample.

25. The data feedback loop system of claim 23, wherein the input comprises feedback loop batching specifications.

26. The data feedback loop system of claim 23, wherein the input comprises ingested data quality specifications.

27. The data feedback loop system of claim 11, wherein the product inference module further comprises: 1) an input selected from a) a deployed model, b) a validated model, c) blood sample data, and d) a combination thereof; and 2) an output selected from a) processed sample molecular data, b) patient test results, c) patient metadata, d) de-identified labeled patient sample data, e) de-identified sample molecular data, and f) a combination thereof.

28. A classification model for disease detection or diagnosis comprising a data feedback loop system, wherein the data feedback loop comprises:
a) a cohort selection and retraining module that selects classes of training samples for a classification model or re-trains the classification models;
b) a product inference module that produces raw data for ingestion into the data feedback loop system; and
c) an external feedback/data collection module that receives data from real-world execution of the classification model, wherein the external feedback/data collection module is operatively linked to a research platform module.

29. The classification model of claim 28, wherein the data feedback loop system further comprises an evaluation/deployment module that productionizes a validated model to prepare for deployment, wherein the evaluation/deployment module is operatively linked between the cohort selection and retraining module and the product inference module.

30. The classification model of claim 28, wherein the data feedback loop system further comprises a data ingestion module that ingests data, wherein the data ingestion module is operatively linked between the external feedback/data collection module and the cohort selection and retraining module.

31. The classification model of claim 28, wherein the classification model comprises a machine learning classifier.

32. The classification model of claim 28, wherein the classification model is trained using a federated learning approach.

33. The classification model of claim 28, wherein the classification model is trained using an active learning approach.

34. A method for re-training a diagnostic classifier, the method comprising:
a) obtaining molecular or clinical data from an individual sample associated with a presence or absence of a specified property of a disease or disorder requiring classification;
b) processing the molecular or clinical data using a data feedback loop system comprising:
i) a research platform module that trains or re-trains a diagnostic classifier;
ii) a production module that produces input data, wherein the production module comprises the diagnostic classifier; and
iii) an external feedback/data collection module that receives data from real-world execution of the diagnostic classifier; and
c) re-training the diagnostic classifier to improve one or more classification metrics of the diagnostic classifier.

35. The method of claim 34, wherein the diagnostic classifier comprises a machine learning classifier.

36. The method of claim 34, wherein the diagnostic classifier is trained using a federated learning approach.

37. The method of claim 34, wherein the diagnostic classifier is trained using an active learning approach.

38. The method of claim 35, wherein the machine learning classifier is trained on a set of training biological samples, wherein the set of training biological samples comprises a first subset of the training biological samples identified as having the specified property and a second subset of the training biological samples identified as not having the specified property, wherein the machine learning classifier provides an output classification of whether the individual sample has the specified property, thereby distinguishing a population of individuals having the specified property.

39. (canceled)

40. The method of claim 34, wherein the specified property comprises a clinically-diagnosed disorder.

41. The method of claim 40, wherein the clinically-diagnosed disorder comprises cancer.

42-77. (canceled)
